(19) United States
(12) Patent Application Publication
(10) Pub. No.: US 2010/0143455 A1
(43) Pub. Date: Jun. 10, 2010

(54) Triazole antifungal agents

(76) Inventors: Nian Wu, North Brunswick, NJ (US); Brian Charles Keller, Antioch, CA (US)

Correspondence Address: Lee Pederson, 712 East Main Street, Sleepy Eye, MN, 56085, US

(21) Appl. No.: 12/584,486

(22) Filed: Sep. 5, 2009

Related U.S. Application Data
(60) Provisional application No. 61/199,821, filed on Nov. 20, 2008.
(60) Provisional application No. 61/191,339, filed on Sep. 8, 2008.

(51) Int. Cl. A01N 25/00; C07D 295/027; A01N 43/60; A01P 3/00

(52) U.S. Cl. 424/450; 544/367; 514/254.01

(57) ABSTRACT

New triazole antifungal agents having C6S7 or S6C7 bridges are disclosed. These triazoles provide alternatives to existing antifungals in terms of formulation, bioavailability and activity.

### CROSS REFERENCE TO RELATED APPLICATIONS

This application claims priority to provisional U.S. Patent Application No. 61/191,339 entitled “Novel Triazole Antifungal Agents” and filed on Sep. 8, 2008 and to provisional U.S. Patent Application No. 61/199,821 entitled “Novel Triazole Antifungal Agents” and filed on Nov. 20, 2008.

### FIELD OF THE INVENTION

This invention relates to antifungal agents. More particularly, this invention relates to new triazole compounds.

### BACKGROUND OF THE INVENTION

Chemical structure 1 shows the chemical structure of the antifungal agent itraconazole, also known by its chemical name of 4-[4-[4-[4-[[2-(2,4-dichlorophenyl)-2-(1H-1,2,4-triazol-1-ylmethyl)-1,3-dioxolan-4-yl]methoxy]phenyl]piperazin-1-yl]phenyl]-2-(1-methylpropyl)-2,4-dihydro-1,2,4-triazol-3-one and/or 4-4-[4-(4-[(3R,5R)-5-(2,4-difluorophenyl)-5-(1H-1,2,4-triazol-1-ylmethyl)tetrahydro-3-furanyl]methoxyphenyl)-piperazino]phenyl-1-[(1S,2S)-1-ethyl-2-hydroxypropyl]-4,5-dihydro-1H-1,2,4-triazol-5-one (all IUPAC names herein are from ChemDraw© (version Ultra 10, CambridgeSoft, Cambridge, Mass., USA).

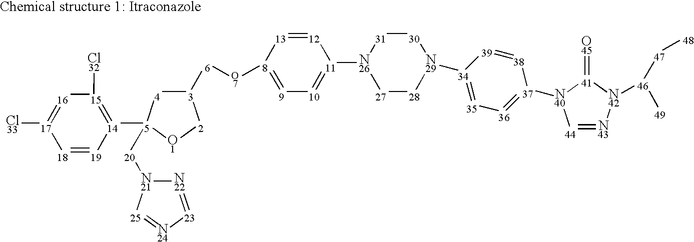

U.S. Pat. No. 5,039,676 discloses difluorophenyl and tetrahydrofuran-substituted derivatives of itraconazole. U.S. Pat. No. 5,403,937 discloses a number of derivatives of 4-(4-(4-(4-((5-((1H-1,2,4-triazol-1-yl)methyl)-5-(2,4-dichlorophenyl)tetrahydrofuran-2-yl)methoxy)phenyl)piperazin-1-yl)phenyl)-1-sec-butyl-1H-1,2,4-triazol-5(4H)-one, including the derivative known as posaconazole, which has a hydroxyhexanyl substitution of sec-butyl at the (N—) 42 position.

As with all azole antifungal agents, itraconazole and posaconazole work principally by inhibition of cytochrome P450 14a-demethylase (P450_{14DM}). This enzyme is in the sterol biosynthesis pathway that leads from lanosterol to ergosterol [Sheehan, D. J., Hitchcock, C. A., and Sibley, C. M., Clin. Microbiol. Rev. 1999, 12:40-79]. Lanosterol 14α-demethylase (P450_{14DM}, CYP51) is a member of the cytochrome P450 superfamily, which catalyzes the removal of the 14-methyl group (C-32) of lanosterol via three successive monooxygenation reactions. The first two of these reactions are conventional cytochrome P450 hydroxylations that produce the 14-hydroxymethyl and 14-carboxyaldehyde derivatives of lanosterol [Trzaskos, J. M., Fischer, R. T., Favata, M. F., J. Biol. Chem. 1986, 261, 16937-16942; Aoyama, Y., Yoshida, Y., Sonoda, Y., Sato, Y., J. Biol. Chem. 1987, 262, 1239-1243]. In the final step, the 14-aldehyde group is eliminated as formic acid with concomitant introduction of a Δ^{14,15} double bond [Aoyama, Y., Yoshida, Y., Sonoda, Y., Sato, Y., J. Biol. Chem. 1989, 264, 18502-18505; Fischer, R. T., Trzaskos, J. M., Magolda, R. L., Ko, S. S., Brosz, C. S., Larsen, B., J. Biol. Chem. 1991, 266, 6124-6132; Shyadehi, A. Z., Lamb, D. C., Kelly, S. L., Kelly, D. E., Schunck, W.-H., Wright, J. N., Corina, D., Akhtar, M, J. Biol. Chem. 1996, 271, 12445-12450]. P450_{14DM} occurs in different kingdoms, such as fungi, higher plants, and animals, with the same metabolic role, i.e., removal of the 14-methyl group of sterol precursors such as lanosterol, obtusifoliol, dihydrolanosterol, and 28-methylene-24,25-dihydrolanosterol [Lamb, D. C., Kelly, D. E., Kelly, S. L., FEES Lett. 1998, 425, 263-265], and is the only known P450 distributed widely in eukaryotes with essentially the same metabolic role [Aoyama, Y.; Noshiro, M., Gotoh, O., Imaoka, S., Funae, Y., Kurosawa, N., Horiuchi, T., Yoshida, Y., J. Biochem. 1996, 119, 926-933; Yoshida, Y., Noshiro, M., Aoyama, Y., Kawamoto, T., Horiuchi, T., Gotoh, O., J. Biochem. 1997, 122, 1122-1128].

In yeasts and fungi P450_{14DM} participates in ergosterol biosynthesis, which is an essential requirement for fungal viability [Lamb, D. C., Kelly, D. E., Venkateswarlu, K., Manning, N. J., Bligh, H. F., Schunck, W. H., Kelly, S. L., Biochemistry 1999, 38, 8733-8738]. The amino acid sequences of P450_{14DM} have been characterized as to substrate specificity by indirect methods for higher plants [Cabello-Hurtado, F., Taton, M., Forthoffer, N., Kahn, R.; Bak, S., Rahier, A. & Werck-Reichhart, D., Eur. J. Biochem. 1999, 262, 435-446], bacteria [Bellamine, A., Mangla, A. T., Nes, W. D. & Waterman, M. R., Proc. Natl. Acad. Sci. U.S.A. 1999, 96, 8937-8942], fungi [Lai, M. H. & Kirsh, D. R., Nucleic Acid Res. 1989, 17, 804; Van Nistelrooy, J. G. M., van der Brink, J. M., van Kan, J. A. L., van Gorcom, R. F. M. & de Waard, M. A., Mol. Gen. Genet. 1996, 250, 725-733], and mammals [Nitahara, Y., Aoyama, Y., Horiuchi, T., Noshiro, M.& Yoshida, Y., J. Biochem. 1999, 126, 927-933; Stromstedt, M., Rozman, D. & Waterman, M. R., Arch. Biochem. Biophys. 1996, 329, 73-81]. Structure-function analysis has not been rigorously studied. No site-directed mutagenesis data are available that could describe key substrate and/or inhibitors binding residues including the heme binding residues. In addition, interactions with redox-partner proteins and/or involvements in electron transfer are not clear.

A pharmacophore model of azole antifungals was proposed initially using miconazole as an example [Talele, T. T & Kulkarni V. M., J. Chem. Inf. Comput. Sci. 1999, 39, 204-210]. A similar model may be applied to itraconazole, where the pharmacophore consists of a trizole ring and a halogenated phenyl ring, both of which are attached to C5 of a 1,3-dioxalane. In both cases the pharmacophores interact with a hydrophobic cavity in the active site of P450_{14DM}. It has been suggested that hydrogen bonds formed between the OH group of substrate and carbonyl and amino groups of the main chain and hydroxyl group of the side chain of P450_{14DM} are essential for orienting the substrate to the correct direction in the active site [Aoyama, Y., Yoshida, Y., Sonoda, Y., Sato, Y., Biochim. Biophys. Acta, 1989, 1006, 209-213; Aoyama, Y., Yoshida, Y., Sonoda, Y., Sato Y., Biochim. Biophys. Acta, 1989, 1001, 196-200; Aoyama, Y., Yoshida, Y., Sonoda, Y., Sato Y., Biochim. Biophys. Acta, 1991, 1081, 262-266; Aoyama, Y., Yoshida, Y., Sonoda, Y. & Sato, Y., Biochim. Biophys. Acta, 1992, 1122, 251-255]. Therefore hydroxyl groups in these antifungals were the essential structures for interacting with the fungal P450_{14DM} protein. A good geometrical fit of pharmacophores and values of the energy difference between the resulting bioactive conformation and the minimum energy for the conformation argued for a reasonable common conformation framework.

A study using a three-dimensional molecular model of P450_{14DM} from Saccharomyces cerevisiae based on homology with P450BM3 was reported [Lewis, D. F. V., Wiseman, A. & Tarbit, M. H., J. Enzyme Inhibit. 1999, 14, 175-192]. The halogenated phenyl ring of ketoconazole was proposed to occupy the same hydrophobic cavity as the 17-alkyl side chain of lanosterol in the model. S378 was identified to interact with the 3-hydroxy group of the substrate, and the 17-alkyl side chain was deep in the same hydrophobic cavity.

Most azole antifungals contain a halogenated phenyl group which has a similar docking mode in the active site of the fungal P450_{14DM} protein. The active site residues interacting with the inhibitors are the same as those interacting with the substrate, i.e., the halogenated phenyl group of the inhibitors is interactive with the same hydrophobic binding cleft as the 17-alkyl chain of substrate. Since the hydrophobic cleft is narrow, the space adjacent to the phenyl group is limited. Thus, bulky substituents larger than a chlorine atom probably produce significant steric clashes and lower binding affinity [Klopman, G. & Ptchelintsev, D., J. Comput.-Aided Mol. Des. 1993, 7, 349-362; Asai, K., Tsuchimori, N., Okonogi, K., Perfect, J. R., Gotoh, O. & Yoshida, Y., Antimicrob. Agents Chemother. 1999, 43, 1163-1169].

Although the side chains of itraconazole, ketoconazole, pramiconazole (under clinical development), and posaconazole are very long, while the side chains of fluconazole, isavuconazole (currently in Phase III clinical trials) and voriconazole are rather short, all of them showed high antifungal activities. The reason is that all of them have the same pharmacophores and the spatial orientations of the pharmacophores are very similar. Even though the side chains of these inhibitors are not the determinants for the anti-fungal activity, they play an important role in adjusting the physicochemical properties of the whole molecule to avoid some dissatisfying side effects and/or improve their pharmacokinetic and pharmacodynamic behavior. The side chains of itraconazole, ketoconazole, pramiconazole, and posaconazole are too long to be accommodated in the active site. However, the long side chains of the inhibitors interact with the residues in the substrate access channel. Especially for itraconazole and posaconazole, the terminal alkyl group of the side chain reaches the entrance of the substrate access channel and interacts with the hydrophobic residues [Talele, T. T. & Kulkarni V. M., J. Chem. Inf. Comput. Sci. 1999, 39, 204-210].

In addition, the distances and orientations between the aromatic ring and other pharmacophores are variable in different azole antifungals which can affect physicochemical properties of the compounds. For example, with voriconazole the chirality at C2 and C3 is important to antifungal activity. The compound shown in Scheme II exhibits significantly higher activities than the other stereoisomers [Tasaka, A., Kitazaki, T., Tsuchimori, N., Matsushita, Y., Hayashi, R., Okonogi, K. & Itoh, K., Chem. Pharm. Bull. 1997, 45, 321-326; Rotstein, D. M., Kertesz, D. J., Walker, K. A. M. & Swinney, D. C., J. Med. Chem. 1992, 35, 2818-2825].

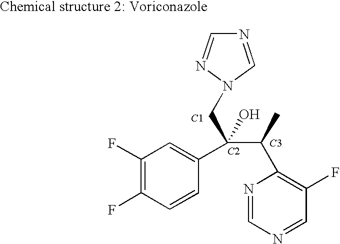

In voriconazole a methyl group attached to C3 was selected as a pharmacophore. The oxygen atom attached to C2 has been suggested to be favorable to antifungal activity as for this and other triazole alcohols such as fluconazole and isavuconazole. These compounds constitute a considerable portion of promising leads in antifungal chemotherapy. These agents are generally more potent, better tolerated, metabolically more stable than the first-generation products which have no hydroxy group at the C2 position [Bartroli, J., Turmo, E., Alguero, M., Boncompte, E., Vericat, M. L., Conte, L., Ramis, J., Merlos, M., Garcia-Rafanell, J. & Form, J., J. Med. Chem. 1998, 41, 1855-1868].

Pramiconazole is a newly developed antifungal, structurally very similar to itraconazole. It is claimed to have a high potential for the treatment of dermatophyte and yeast infections of the skin. However whether pramiconazole is more active than its parent agent (itraconazole) is not known. In vitro experimental results indicate a slow enzyme-mediated disappearance of pramiconazole. In addition, in a clinical trial to investigate whether pramiconazole is converted into a more potent active metabolite in humans blood samples from healthy volunteers, no active metabolite present in blood samples was found after oral dosing [Ausma J., Pennick G., Bohets H., van de velde V., Borgers M. & Fothergill A., Acta dermato-venereologica 2007, 87, 22-26].

Given the rise in severe fungal infections in immuno-suppresed human patients and the limitations of current anti-fungal agents, it is desirable to develop new and improved agents and compositions for treatment.

### BRIEF SUMMARY OF THE INVENTION

New triazole antifungal agents having C6S7 or S6C7 bridges are disclosed. These triazoles provide alternatives to existing antifungals in terms of formulation, bioavailabilty and activity.

### BRIEF DESCRIPTION OF THE FIGURES

The accompanying drawings, which are incorporated into and constitute a part of this specification, illustrate one or more embodiments of the present invention and, together with the detailed description, serve to explain the principles and implementations of the invention.

FIG. 1 shows pharmacokinetic profiles of posaconazole and equaconazol formulations after intravenous dosing.

FIG. 2 shows pharmacokinetic profiles of posaconazole and equaconazole formulations after oral dosing.

### DETAILED DESCRIPTION OF THE INVENTION

Embodiments of the present invention are described herein in the context of novel triazole antifungal agents. Those of ordinary skill in the art will realize that the following detailed description of the present invention is illustrative only and is not intended to be in any way limiting. Reference will now be made in detail to implementations of the present invention.

In comparison with itraconazole, posaconazole is said to be a significantly more potent inhibitor of sterol C14 demethylation, particularly in Aspergillus [Munayyer, H., K. J. Shaw, R. S. Hare, B. Salisbury, L. Heimark, B. Pramanik, & J. R. Greene. 1996. “SCH 56592 is a potent inhibitor of sterol C14 demethylation in fungi.” 36th Interscience Conference on Antimicrobial Agents and Chemotherapy]. The fluorine substituents are less bulky than chlorine and have less of a steric barrier and higher binding affinity, and the 1,3-dioxalane junction between the pharmacophore and side chain is probably more favorable than the tetrahydrofuran in the molecules for interacting with the active sites of P450_{14DM}. The C6-O7 bridge is the same in both molecules.

The present invention includes altering the C6 and 07 positions to generate an extended pharmacophore with geometrically favorable physicochemical properties for these agents which should be considered in designing new antifungals to improve their selectivity to P450_{14DM} of fungi. In particular S6-C7 and C6-S7 bridges are employed. Since there is very little difference in electronegativity between sulfur and carbon, an S—C bond is less polar than a C—O bond and it does not form hydrogen bonds. Therefore it has no interference to the hydrophobic interaction between the pharmacophore of the antifungal agents and the active site of P450_{14DM}.

This invention relates to novel triazole derivatives, which may be referred to herein as equaconazoles, which have extended pharmacophores and improved physicochemical properties of antifungal activity and are useful in the medical treatment of fungal infections in humans and animals. This invention includes several derivatives having the general structure:

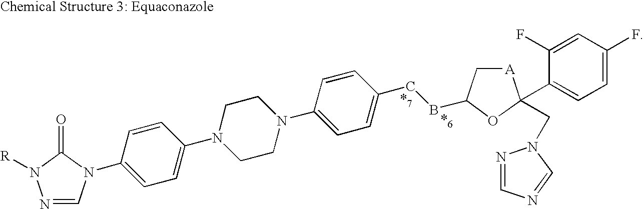

where A is CH₂ or oxyl, one of either B or C is CH₂ and the other is thiol (sulfur), and specific R groups are listed in Table 1.

TABLE 1

| | | | Molecular |
|---|---|---|---|
| Symbol | IUPAC | Substituent | Weight |
| Rₐ | sec-butyl | 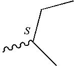 | 72.15 |
| R_{b} | Isopentanyl | 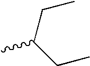 | 86.18 |
| R_{c} | Isopropyl | 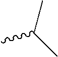 | 58.12 |
| R_{d} | 2-isopropiono-1-nitrile | 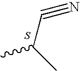 | 69.11 |
| Rₑ | 3-isobutan-2-ol | 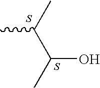 | 88.15 |
| R_{f} | 3-isopentan-2-ol | 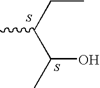 | 102.18 |
| R_{g} | 2-isobut-1-ene | 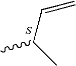 | 70.13 |

In Table 1, “S” indicates the preferred conformation of chiral centers, though the invention includes alternate conformations.

R groups may affect formulation, bioavailability, and activity of equaconazoles. Based on antifungal activity testing described below in Example 41, specifically preferred R groups of those listed in Table 1 are sec-butyl and/or 3-isopentan-2-ol. The most preferred specifically disclosed R group is sec-butyl. The compounds of the present invention are meant to comprise other R groups that have not specifically been disclosed. Generally, these other R groups will be comprised of carbon, oxygen, nitrogen and hydrogen. Their molecular weight will preferably be between about 58 and 200, and more preferably between about 58 and 102.

Equaconazoles have been synthesized as described herein. Testing has shown that they have significant antifungal activities, in some cases exceeding the activity of commercially available agents.

It is useful to employ Chemical Reaction Scheme 1 to describe the chemical synthesis of the S6-C7 and C6-S7 bridges of equaconazoles, though formation of the bridge is not the final reaction step in the synthetic methods used herein. In the examples, the four specific variants of Compound A are completely synthesized prior to the bridge-forming reactions of Chemical Reaction Scheme 1, while the many variants of Compound B are not completely synthesized until after the bridge is formed. Examples 8-11 describe in detail the syntheses of the two specific types of bridges. In each of those examples, a precursor to Compound B is reacted with Compound A to form the bridge. The end of the equaconazole molecule containing the R group is completed only after the bridge is formed. Still, Chemical Reaction Scheme 1 is a useful tool to conceptualize the variations of equaconazoles included in the invention.

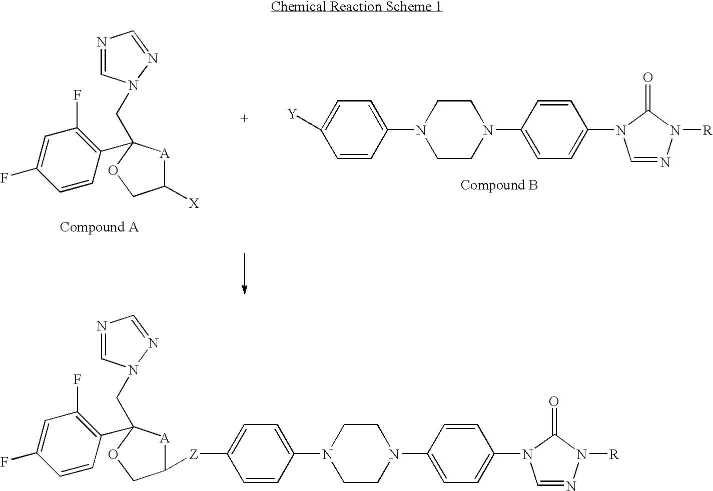

where A and X and Y and Z are listed in Table 2.

TABLE 2

| Formula | X (Compound A) | Y (compound B) | Z |
|---|---|---|---|
| I (D) | CH₂SH | OH | —CH₂S— |
| A = O | | | (C6S7 bridge) |
| I (T) | CH₂SH | OH | —CH₂S— |
| A = CH₂ | | | (C6S7 bridge) |
| II (D) | SH | CH₂OH | —SCH₂— |
| A = O | | | (S6C7 bridge) |
| II (T) | SH | CH₂OH | —SCH₂— |
| A = CH₂ | | | (S6C7 bridge) |

Intermediate Compound A may alternatively comprise either a tetrahydrofuran (T) ring or a dioxalon (D) ring. Synthesis of the dioxolan version of compound A where X is —SH is described in Examples 1 and 2 herein. Synthesis of a precursor to the tetrahydrofuran version of compound A where X is —OH is described in Examples 3 to 5. Conversion of —OH to —SH as described in example 7 completes the synthesis of the tetrahydrofuran version of compound A where X is —SH. Precursors to compound A (both dioxolan or tetrahydrofuran versions) where X is CH₂OH are commercially available (Fulcrum Scientific, England, UK and ChemPacific Corporation, Baltimore, Md.). Conversion of —OH to —SH as described in example 7 completes the synthesis of these variants. Alternatively, they may be prepared as described in Examples 1 to 5, and then converting the —OH to —CH₂SH.

Examples 8-11 describe the syntheses of four intermediates, each starting with one of the four variants of Compound A, where the bridge is formed. Example 12 describes addition of a triazolone ring to the Compound B portion each of these four intermediates. Final synthetic steps for each of the 28 variants of equaconazole specifically disclosed herein, including addition of R groups, are described in examples 13-40.

The four main variants of equaconazole are shown below. These variants correspond to the four intermediates whose syntheses are described in examples 8-11. In the formula labels for structures 4-7, the Roman numeral indicates the bridge structure based on Table 2. “I” indicates that the compound comprises a C6-S7 bridge and “II” indicates that the compound comprises a S6-C7 bridge. “D” indicates that the compound comprises a dioxolan ring and “T” indicates that the compound comprises a tetrahydrofuran ring. “R” indicates an as yet unspecified R group.

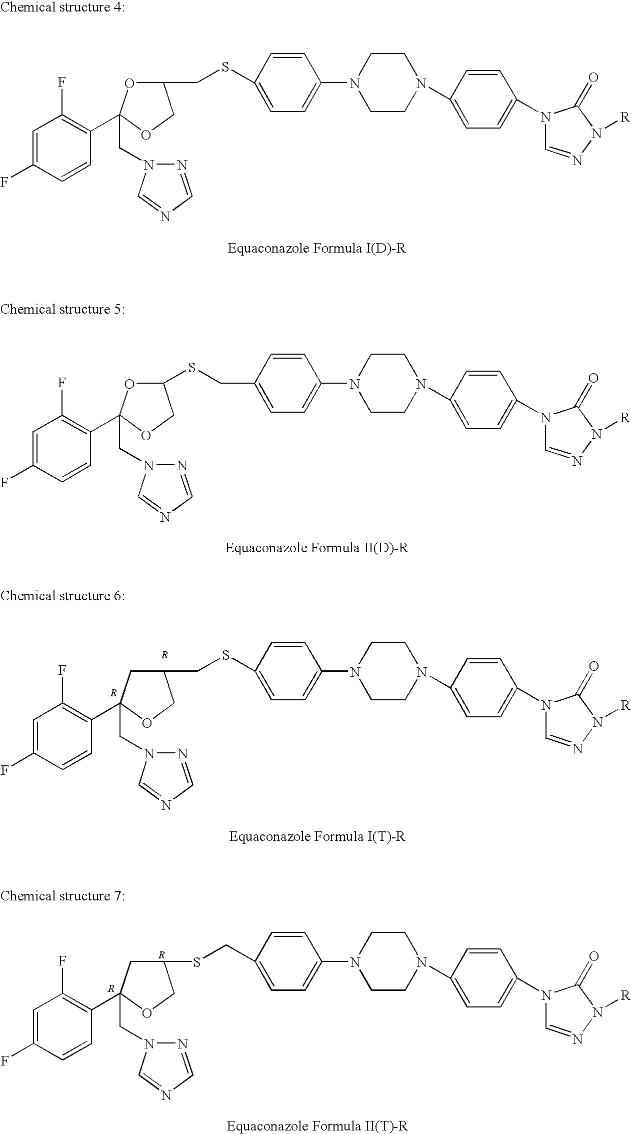

The starting materials of compounds used to prepare equaconazoles are commercially available. Some synthetic steps employed conventional processes or published methods which are described in U.S. Pat. Nos. 4,916,134; 5,039,676 and 5,116,844, which are hereby incorporated by references. Of course, modifications were needed to create the novel compounds described herein.

In general, the synthesis of the intermediate material of compound A (1-D) started from 2,4-di-difluoroacetophenone and 2-hydroxymethyl-1,3-propanediol. The reaction was performed in a benzene-1-butanol medium with azeotropic removal of water in the presence of a catalytic amount of p-toluenesulfonic acid. Without isolation, the formed ketal was brominated at 30° C. to bromo ketal. Benzoylation of bromo ketal in pyridine afforded the ester as a cis/trans mixture, from which the cis form could be isolated by crystallization from EtOH. The pure trans isomer could be obtained by liquid chromatography of the reactant liquor. Coupling of bromo ketal in dry DMA with triazole gave 2-((1H-1,2,4-triazol-1-yl)methyl)-2-(2,4-difluorophenyl)-1,3-dioxolan-4-yl benzoate. The ester was saponified by refluxing with NaOH in dioxane-water medium to a precursor to compound A where X was —OH. This precursor could be further converted to forms of compound where X was either —S or —CH₂S.

Though bridge formation is described using the reactive groups X and Y shown in Table 2, other synthetic methods are possible. For example, the bridge in Formula I compounds can be constructed from a reaction where X is —CH₂Br and Y is —SH. Similarly, the bridge in Formula II compounds can be constructed from a reaction where X is —Br and Y is —CH₂SH.

The novel triazole antifungal agents of the present invention are useful for the treatment of fungal infections in mammals. Depending on the infection to be treated or prevented, they may be administered by oral solution, oral capsule, topically or through intravenous (IV) administration.

The antifungal agents of the present invention are typically formulated with one or more pharmaceutically acceptable carriers that are known in the art. In a preferred mode, the agents are formulated into liposomes. They may be formulated as spontaneously forming liposomes described in U.S. Pat. No. 6,958,160, which is hereby incorporated by reference. In addition to oxyl linkage described in the '160 patent, the DAG-PEG lipids may have a variety of chemical linkages between the glycerol backbone and the PEG chain as shown in Table 3. Preferred diacylglycerol-polyethyleneglycol (DAG-PEG) lipids include PEG-12-N₃-GDO, PEG-12-N₃-GDM, PEG-12-N₃-GDLO, PEG-12-N₃-GDP, PEG-12-Ac₂-GDO, PEG-12-Ace-GDM or any combination thereof. GDO means glycerol dioleate, GDM means glycerol dimyristate, GDLO means glycerol dilinoleate, GDL means glycerol dilaurate, and GDP means glycerol dipalmitate. The numeral after the PEG means the number of subunits in the PEG chain. For example, PEG-12 refers to a PEG chain having 12 subunits. Notations such as N₃ in PEG-12-N₃-GDO refer to the linker from Table 3 that connects the PEG chain to the glycerol backbone. Formulations of the agents as spontaneously forming liposomes result in increased bioavailability when administration is by an IV route.

TABLE 3
Linkers

| No | Symbol | X |
|---|---|---|
| 1 | N₁ | 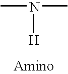 |
| 2 | N₂ | 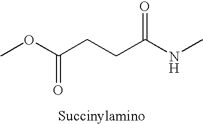 |
| 3 | N₃ | 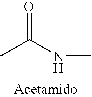 |
| 4 | N₄ | 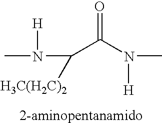 |
| 5 | N₅ | 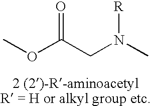 |
| 6 | S₁ | 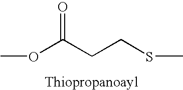 |
| 7 | S₂ | 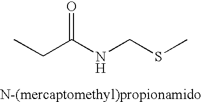 |
| 8 | S₃ | 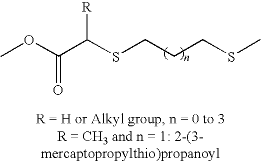 |
| 9 | S₄ | 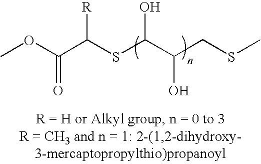 |
| 10 | Ac₁ | 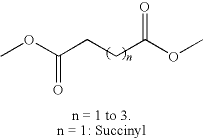 |
| 11 | Ac₂ | 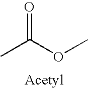 |
| 12 | Ac₃ | 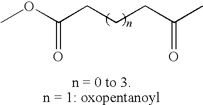 |
| 13 | N₆ | 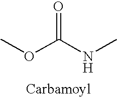 |
| 14 | N₇ | 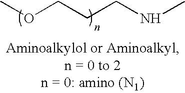 |
| 15 | N₈ | 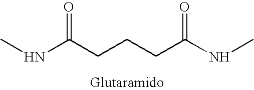 |
| 16 | S₅ | 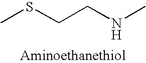 |
| 17 | S₆ | 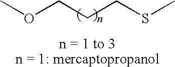 |
| 18 | S₇ | 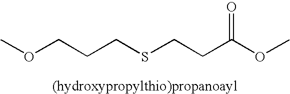 |
| 19 | S₈ | 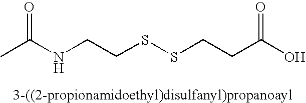 |
| 20 | S₉ | 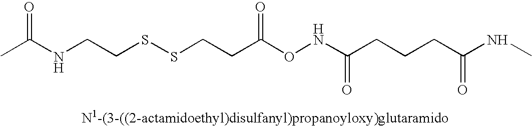 |
| 21 | S₁₀ | 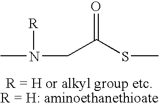 |
| 22 | Ac₄ | 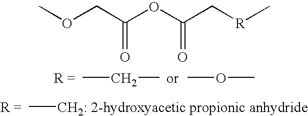 |

When the R group of equaconazoles shown in Chemical structure 3 contains a hydroxyl group, as when R is 3-isobutan-2-ol or 3-isopentan-2-ol, it is possible to convert the hydroxyl group to an ester, ether, or biodegradable salt without departing from basic invention described herein. In such cases derivatives may be bioconverted to the hydroxyl form after administration.

It an object of the invention to provide new agents for the effective prevention and treatment of fungal infections in mammals. It is a further objective to provide pharmaceutical formulations for such prevention and treatment. It is a further objective to provide a method of treatment of fungal infections in mammals.

In one aspect the invention includes compounds represented by the formula shown in Chemical structure 3 where A is CH₂ or oxyl, one of B and C is thiol and the other is CH₂, and R is selected from the group consisting of sec-butyl, isopentanyl, isopropyl, 2-isopropriono-1-nitrile, 3-isobutan-2-ol, 3-isopentan-2-ol, and 2-isobut-1-ene. The invention also includes esters of these compounds, where R is selected from the group consisting of 3-isobutan-2-ol, 3-isopentan-2-ol. Such esters are convertible in vivo into OH, thereby forming the original compound. The invention also includes pharmaceutically acceptable salts of the compounds, where R is either 3-isobutan-2-ol or 3-isopentan-2-ol. As with the esters, such salts are convertible in vivo into OH.

In another aspect the invention is a pharmaceutical composition for treating or preventing fungal infection comprising an antifungally effective amount of a compound shown in chemical structure 3 together with a pharmaceutically acceptable carrier therefore. The pharmaceutically acceptable carrier may be a DAG-PEG. The DAG-PEG may be selected from the group consisting of PEG-12 GDO, PEG-12 GDM, PEG-23 GDP, PEG-12 GDS and PEG-23 GDS. Generally, preferred DAG-PEGs here and elsewhere in this patent include those with any type of chemical linkage between the PEG chain and the glycerol backbone, whether specified or not.

In another aspect the invention is a method of treating and/or preventing a fungal infection in a mammal comprising administering an antifungally effective amount of a compound shown in chemical structure 3 sufficient for such treating or preventing. The method may employ a means selected from the group consisting of oral capsule, oral solution, topical solution, and intravenous suspension.

While the preferable compounds in the present invention are thermally stable, as well as physically and chemically compatible with commonly used pharmaceutical excipients, they are water insoluble triazole compounds which result in low and variable bioavailability in animals if administered without a proper formulation. Liposomal formulations using diacylglycerol-polyethylene glycol were developed to enhance the bioavailability and to reduce the food effect. Therefore, in another aspect the invention includes a method of making a pharmaceutical composition for treating or preventing a fungal infective comprising combining a compound shown in chemical structure 3 with a DAG-PEG and an aqueous solution to form liposomes. The DAG-PEG me be selected from the group consisting of PEG-12 GDO, PEG-12 GDM, PEG-23 GDP, PEG-12 GDS and PEG-23 GDS.

In yet another aspect the invention includes a compound represented by the formula shown in chemical structure 3 where A is CH₂ or oxyl, one of B and C is thiol and the other is CH₂, and R has a molecular weight below about 200. This aspect includes esters and pharmaceutically acceptable salts of the compounds where R comprises an OH group. This aspect also includes the compound formulated with a pharmaceutically acceptable carrier. The pharmaceutically acceptable carrier may be a DAG-PEG. The DAG-PEG may be selected from the group consisting of PEG-12 GDO, PEG-12 GDM, PEG-23 GDP, PEG-12 GDS and PEG-23 GDS.

### Example 1

### Synthesis of 2-(bromomethyl)-2-(2,4-difluorophenyl)-4-(ethylthio)-1,3-dioxolane

To a stirred solution of 1-(2,4-difluorophenyl)-1-ethanone (1.0 mol) in 240 mL of n-butanol, 1.2 moles of bromine was added dropwise at room temperature. After stirring for 1 hour at room temperature, the reaction mixture was charged successively with 1.2 moles of 3-ethylsulfanyl-propane-1,2-diol, 540 mL of anhydrous benzene and 0.03 moles of p-toluenesulfonic acid monohydrate. The resulting mixture was stirred under refluxing overnight with the equipment of water-separator for 2 hours. After solvent was evaporated, under vacuo, the residue was dissolved in dichloromethane. The solution was washed with a diluted sodium hydroxide solution, followed with water 3 times. After dried over Na₂SO₄, The resulting mixture was filtered and evaporated. The residue was distilled to afford 2-(bromomethyl)-2-(2,4-difluorophenyl)-4-(ethylthio)-1,3-dioxolane (Chemical Structure 8) as colorless oil (˜80%) as shown in Chemical Structure 8.

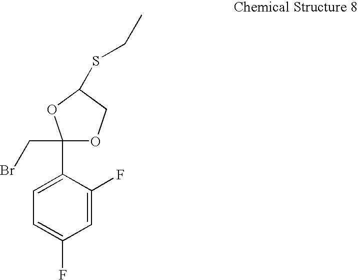

### Example 2

### Synthesis of 2-((1H-1,2,4-triazol-1-yl)methyl)-2-(2,4-difluorophenyl)-1,3-dioxolane-4-thiol

Sodium triazole was prepared in-situ by azeotropically distilling a mixture of 1,2,4-triazole (0.5 mol), sodium hydroxide solution (50%, 35.2 mL), toluene, (250 mL) and dimethylsulfoxide (250 mL) to a Karl Fisher water content less than 0.4% (by a Karl Fisher titration). The solution was cooled to 25° C., then a anhydrous toluene solution of 2-(bromomethyl)-2-(2,4-difluorophenyl)-4-(ethylthio)-1,3-dioxolane (Chemical Structure 8, 1.8 mol) was added. The temperature was increased to 75° C. and held at that temperature to reaction completion (monitored by TLC). Then the reaction mixture was cooled to 35° C. and quenched with dilute sodium hydroxide aqueous solution slowly to keep the temperature below 45° C. After the resulting mixture was cooled to 25 to 35° C., water and toluene were added. After the phases were separated, the aqueous phase was washed with toluene 3 to 5 times. The combined organic phases were concentrated under a maximum temperature of 70° C. The ethyl group of the thioester (1 mol) was removed by treated the resulting residue with sodium methoxide (approximately 8 mol) in dimtheylformamide (300 mL). The reaction mixture was stirred under nitrogen over night at 120° C. After cooling to room temperature, the reaction was quenched by adding methyl iodide (5 mol) under constantly stirring. Then the mixture was poured into water (350 mL) and extracted with tert-butyl methyl ether (100 mL×3). The organic phase was washed with saturated sodium sulphate solution and condensed. The residue was dissolved in warm toluene and washed with dilute hydrochloric acid aqueous solution, up to two times. After filtration of the precipitate and crystallization from isopropanol/Isopropyl ether (8/2, v/v), the D form of Chemical Structure A where X is —SH was obtained in ˜65% yield (Chemical Structure 9).

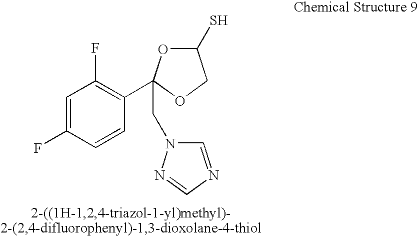

### Example 3

### Synthesis of 5-(2,4-difluorophenyl)-5-ethyltetrahydrofuran-3-ol

Diphenyl diselenide (1 mmol) was dissolved in CH₂Cl₂ (8 mL) at 0° C., then silver trifluoromethanesulfonate (1 mmol) was added. After 10 min, 1-(but-1-en-2-yl)-2,4-difluorobenzene (2.5 mmol) and isopropyl glycolate (1.2 mmol) were added at −78° C. The reaction temperature was left to slowly warm up to −50° C. over 30 min. The resulting white suspension was stirred for 1 h and then quenched with water (30 mL). The mixture was filtered through Celite and extracted with CH₂Cl₂ (3×15 mL). The organic layer was dried over Na₂SO₄ and condensed. The crude ester was purified by flash chromatography (light petroleum ether/dichloromethane 80:20 to 60:40 as eluent). The ester (1 mmol) was dissolved in toluene (8 mL) and treated at −78° C. with 1.1 equiv of Diisobutylaluminium hydride (1.5 M in toluene) under N₂. After stirring under N₂ for 3 h, 20 ml, of a 7% HCl aqueous solution was added, the mixture was continuously stirred at room temperature for 3 h. Then the reaction mixture was extracted with CH₂Cl₂ (3×15 mL). The combined organic layer was dried over Na₂SO₄, and condensed under reduced pressure. The residue (aldehyde) was used without further purification. To the solution of aldehyde (0.26 g, 0.5 mmol) in toluene (5 mL), tributylstannane (0.27 mL, 1 mmol) was added as one portion, followed by a catalytic amount of azobisisobutyronitrile. The reaction mixture was refluxed under N₂ for 2 h. The progress of the reaction was monitored by TLC. The solvent was removed under reduced pressure. The crude mixture was purified by flash chromatography (light petroleum ether/diethyl ether 70:30 to 55:45 as eluent). The desired Chemical Structure 10 was obtained in a yield of 57% to 65%.

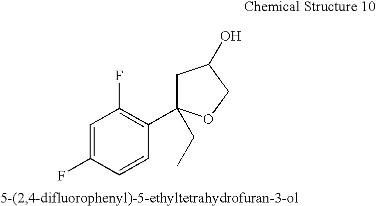

### Example 4

### Synthesis of 5-(2,4-difluorophenyl)-5-propyltetrahydrofuran-3-yl benzenesulfonate

The solution of 5-(2,4-difluorophenyl)-5-ethyltetrahydrofuran-3-ol (1.5 moles) from Example 3 was azeotropically dried with toluene and combined with diazabicyclo[2,2,2]octane (DABCO). While maintaining the reaction temperature below 25° C., a toluene solution (about 140 mL) of p-chlorobenzenesulfonyl chloride (1.4 moles) was charged. On reaction completion, the reaction was then slowly combined with a dilute sodium hydroxide solution while maintaining the reaction temperature below 25° C. Cold tetrahydrofuran (approximately 100 mL) was added and the mixture was aged until the excess sulphonyl chloride was consumed. The reaction was acidified to remove DABCO, followed by a water wash to remove excess acid. The organic phase was washed with dilute aqueous sodium bicarbonate (repeated as needed to achieve a neutral pH), and finally with water. The organic phase was concentrated to a reduced vacuo. After distillation with methanol to displace residual toluene, the residue was filtered, washed with cold methanol and dried below 50° C. The yield of desired Chemical Structure 11 was around 80%.

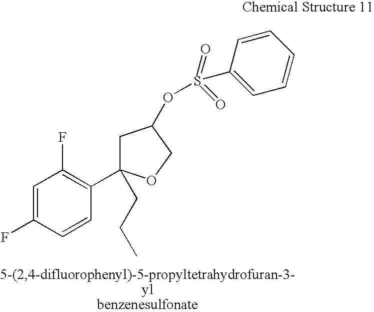

### Example 5

### Synthesis of 5-(2-(1H-1,2,4-thazol-1-yl)ethyl)-5-(2,4-difluorophenyl)tetrahydrofuran-3-ol

To the solution of 5-(2,4-difluorophenyl)-5-propyltetrahydrofuran-3-yl benzenesulfonate (1.5 mol) in acetonitrile, a mixture of iodine (3 moles) and sodium phosphate tribasic (0.5 mole) in dimethylformamide (about 250 mL) was added gradually at −30 to −20° C. After stirring the resulting mixture at this temperature for 1 hour, it was allowed to slowly warm up to room temperature to complete the formation of 5-(2,4-difluorophenyl)-5-(2-iodoethyl)tetrahydrofuran-3-yl benzenesulfonate. The reaction mixture was quenched with aqueous sodium bisulfite under the temperature between 5 to 22° C. The aqueous phase was extracted with tert-butyl methyl ether. The combined organic phases were washed with aqueous sodium hydroxide once, followed by water twice. The solvent was removed under reduced pressure at pot temperature less than 80° C. The oily residue containing 5-(2,4-difluorophenyl)-5-(2-iodoethyl)-tetrahydrofuran 3-yl benzenesulfonate was dissolved in tert-butyl methyl ether and concentrated under reduced pressure again under 80° C. It was redissolved in tert-butyl methyl ether and the solution was carried directly onto the next stage without further purification.

Sodium triazole was prepared in-situ by azeotropically distilling a mixture of 1,2,4-triazole (1.8 moles), sodium hydroxide solution (50%, 180 mL), toluene, (about 120 mL) and dimethylsulfoxide (120 mL) to a Karl Fisher water content less than 0.5%. The solution was cooled to 25° C., then a tert-butyl methyl ether solution of 5-(2,4-difluorophenyl)-5-(2-iodoethyl)tetrahydrofuran-3-yl benzenesulfonate (1 mol) was added. Then tert-butyl methyl ether was removed by distillation under maximum pot temperature of 105° C. and held at that temperature until reaction completion. The reaction was cooled to 35° C. and quenched with dilute sodium hydroxide under maximum temperature of 45° C. The quenched mixture was cooled to 35° C., followed by the addition of water and tert-butyl methyl ether. After the phases were separated, the aqueous phase was washed with tert-butyl methyl ether couple of times. The combined organic phases were concentrated under maximum temperature of 70° C. The residue was extracted with hot toluene 2-3 times. The organic phase was concentrated and dried under vacuum to give the desired product 12 in the yield of 65 to 70% (Chemical Structure 12).

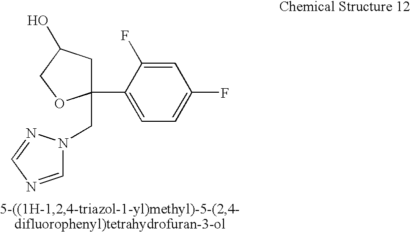

### Example 6

(2-((1H-1,2,4-triazol-1-yl)methyl)-2-(2,4-difluorophenyl)-1,3-dioxolan-4-yl)methanol (Chemical Structure 13) and (5-((1H-1,2,4-triazol-1-yl)methyl)-5-(2,4-difluorophenyl)-tetrahydrofuran-3-yl)methanol (Chemical Structure 14) are commercially available (Fulcrum Scientific, England, UK; ChemPacific Corporation, Baltimore, Md.; Beijing Huikang Boyuan Chemical Tech Co., Ltd. Beijing, China). They were purchased to apply into the preparation of Trizolones in Examples 7 and 12 without further purification.

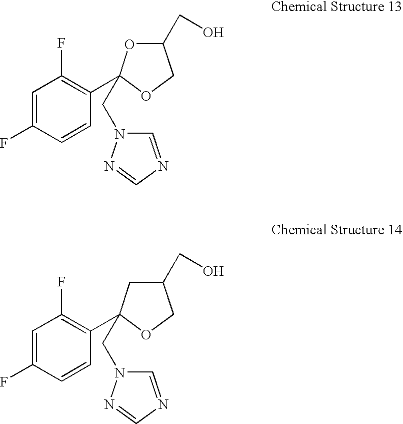

### Example 7

### General Procedure of the Conversion of Hydroxyl (X) Group to Thiols

A mixture of the substrate (1 mole, Chemical Structure A; D or T form in Scheme 1) and a 250 mL of solvent mixture of hexane and water (1/2, v/v) was placed into a high-pressure reactor, followed by the addition of 0.05 mol of dicobalt octacarbonyl (CO₂(CO)₈. After purging with carbon monoxide twice, the reactor was charged with hydrogen sulfide (26 atm) and carbon monoxide (48 atm). The reaction mixture was stirred for 10 h at 150° C. under this pressure. Then the mixture was cooled to room temperature, and the pressure was carefully released in fume hood. The pale brown homogeneous mixture was poured into a beaker and allowed to stand in air for a few minutes. During this time, the mixture turned black and some precipitate was formed. The crude product was washed with hexane (3×50 mL), ether (3×50 mL), and ethanol (3×50 mL), and the washings were added to the reaction mixture. The mixture was treated with Celite and then magnesium sulfate, filtered using additional ether (150 mL) and then hexane (100 mL), and concentrated to afford oil. The oil was analyzed by gas chromatography-mass spectrometry. Products were isolated by fractional distillation or flash chromatography,

### Example 8

### Synthesis of 1-(4-(4-((5-((1H-1,2,4-triazol-1-yl)methyl)-5-(2,4-difluorophenyl)tetrahydrofuran-3-ylthio)methyl)phenyl)piperazin-1-yl)ethanone

Anhydrous zinc iodide (0.5 mol) was added to a solution of 1-{4-[4-(hydroxymethyl)phenyl]-piperazin-1-yl}ethan-1-one (1 mol) in anhydrous 1,2-dichloroethane (350 mL), followed by the addition of 5-((1H-1,2,4-triazol-1-yl)methyl)-5-(2,4-difluorophenyl)-tetrahydrofuran-3-thiol (1.2 mmol). The resulting mixture was stirred at room temperature for 40 min until the reaction was completed. The reaction was quenched with water (10 mL). After extraction with dichloromethane (2×10 mL), the combined organic layer was washed with brine and dried over Na₂SO₄, then the solvent was evaporated at reduced pressure. The residue was purified by flash chromatography over silica gel (70-230 mesh), with 1:100 ethyl acetate-hexane as eluent to afford 1-(4-(4-((5-((1H-1,2,4-triazol-1-yl)methyl)-5-(2,4-difluorophenyl)-tetrahydrofuran-3-ylthio)methyl)phenyl)piperazin-1-yl)ethanone (91%). Excess thiol can be washed out during the workup by treatment with 1 N NaOH as desired.

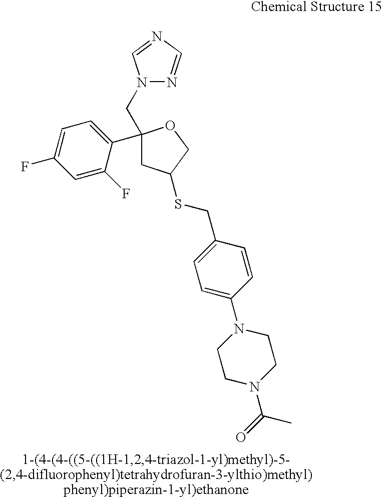

### Example 9

### Synthesis of 1-(4-(4-((2-((1H-1,2,4-triazol-1-yl)methyl)-2-(2,4-difluorophenyl)-1,3-dioxolan-4-ylthio)methyl)phenyl)piperazin-1-yl)ethanone

Anhydrous zinc iodide (0.5 mol) was added to a solution of 1-{4-[4-(hydroxymethyl)-phenyl]-piperazin-1-yl}ethan-1-one (1 mol) in anhydrous 1,2-dichloroethane (350 mL), followed by the addition of 2-((1H-1,2,4-triazol-1-yl)methyl)-2-(2,4-difluorophenyl)-1,3-dioxolane-4-thiol (1.2 mmol). The resulting mixture was stirred at room temperature for 40 min until the reaction was completed. The reaction was quenched with water (10 mL). After extraction with dichloromethane (2×10 mL), the combined organic layer was washed with brine and dried over Na₂SO₄. then the solvent was evaporated at reduced pressure. The residue was purified by flash chromatography over silica gel (70-230 mesh) with ethyl acetate-hexane (1/1, v/v) as eluent to afford 1-(4-(4-((2-((1H-1,2,4-triazol-1-yl)methyl)-2-(2,4-difluorophenyl)-1,3-dioxolan-4-yloxy)methyl)phenyl)piperazin-1-yl)ethanone (93%). Excess thiol can be washed out during the workup by treatment with 1 N NaOH as desired.

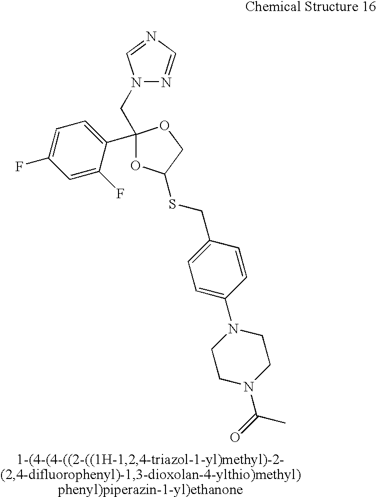

### Example 10

### Synthesis of 1-(4-(4-((5-((1H-1,2,4-triazol-1-yl)methyl)-5-(2,4-difluorophenyl)tetrahydrofuran-2-yl)methylthio)phenyl)piperazin-1-yl)ethanone

Anhydrous zinc iodide (0.5 mol) was added to a solution of 1-acetyl-p-hydroxyphenyl-piperazine (1 mol) in anhydrous 1,2-dichloroethane (350 mL), followed by the addition of (5-((1H-1,2,4-triazol-1-yl)methyl)-5-(2,4-difluorophenyl)tetrahydrofuran-3-yl)methanethiol (1.2 mmol). The resulting mixture was stirred at room temperature for 40 min until the reaction was completed. The reaction was quenched with water (10 mL). After extraction with dichloromethane (2×10 mL), the combined organic layer was washed with brine and dried over Na₂SO₄, then the solvent was evaporated at reduced pressure, the residue purified by flash chromatography over silica gel (70-230 mesh) with ethyl acetate-hexane (1/1, v/v) as eluent to afford 1-(4-(4-((5-((1H-1,2,4-triazol-1-yl)methyl)-5-(2,4-difluorophenyl)tetrahydrofuran-2-yl)methylthio)phenyl)piperazin-1-yl)ethanone (88%). Excess thiol can be washed out during the workup by treatment with 1 N NaOH as desired.

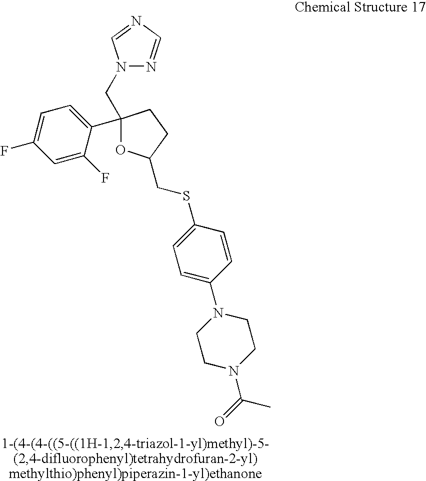

### Example 11

### Synthesis of 1-(4-(4-(4-((2-((1H-1,2,4-triazol-1-yl)methyl)-2-(2,4-difluorophenyl)-1,3-dioxolan-4-yl)methylthio)phenyl)piperazin-1-yl)ethanone

Anhydrous zinc iodide (0.5 mol) was added to a solution of 1-acetyl-p-hydroxyphenyl-piperazine (1 mol) in anhydrous 1,2-dichloroethane (350 mL), followed by the addition of (2-((4(1H-1,2,4-triazol-1-yl)methyl)-2-(2,4-difluorophenyl)-1,3-dioxolan-4-yl)methanethiol (1.2 mmol). The resulting mixture was stirred at room temperature for 40 min until the reaction was completed. The reaction was quenched with water (10 mL). After extraction with dichloromethane (2×10 mL), the combined organic layer washed with brine and dried over Na₂SO₄, then the solvent was evaporated at reduced pressure. The residue purified by flash chromatography over silica gel (70-230 mesh) with ethyl acetate-hexane (1/1, v/v) as eluent to afford 1-(4-(4-((2-((1H-1,2,4-triazol-1-yl)methyl)-2-(2,4-difluorophenyl)-1,3-dioxolan-4-yl)methylthio)phenyl)piperazin-1-yl)ethanone (90%). Excess thiol can be washed out during the workup by treatment with 1 N NaOH as desired.

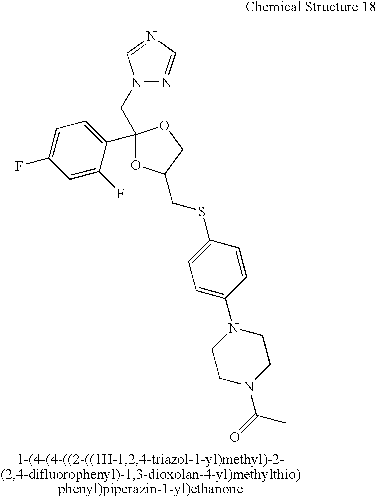

### Example 12

### Synthesis of Trizolones

Trizolones can be prepared by from several ways [Heeres, J., Backx, L. J. J. and Van Cutsem, J.; Antimycotic azoles. 7. Synthesis and antifungal properties of a series of novel triazol-3-ones; J Med Chem 27 (1984) 894-900]. In general, the substances prepared from Example 8 to 11 were desacetylated in the presence of NaOH in refluxing butanol to give the corresponding piperazine (I). The condensation of (I) with 4-chloronitrobenzene in the presence of K₂CO₃ in hot DMSO afforded the nitro Chemical Structure (II), which was reduced with H₂ in the presence of Pt/C in ethyleneglycol to give the corresponding aniline (III). The reaction of (IV) with phenyl chloroformate in the presence of pyridine in CHCl₃ gave the phenylcarbamate (V).

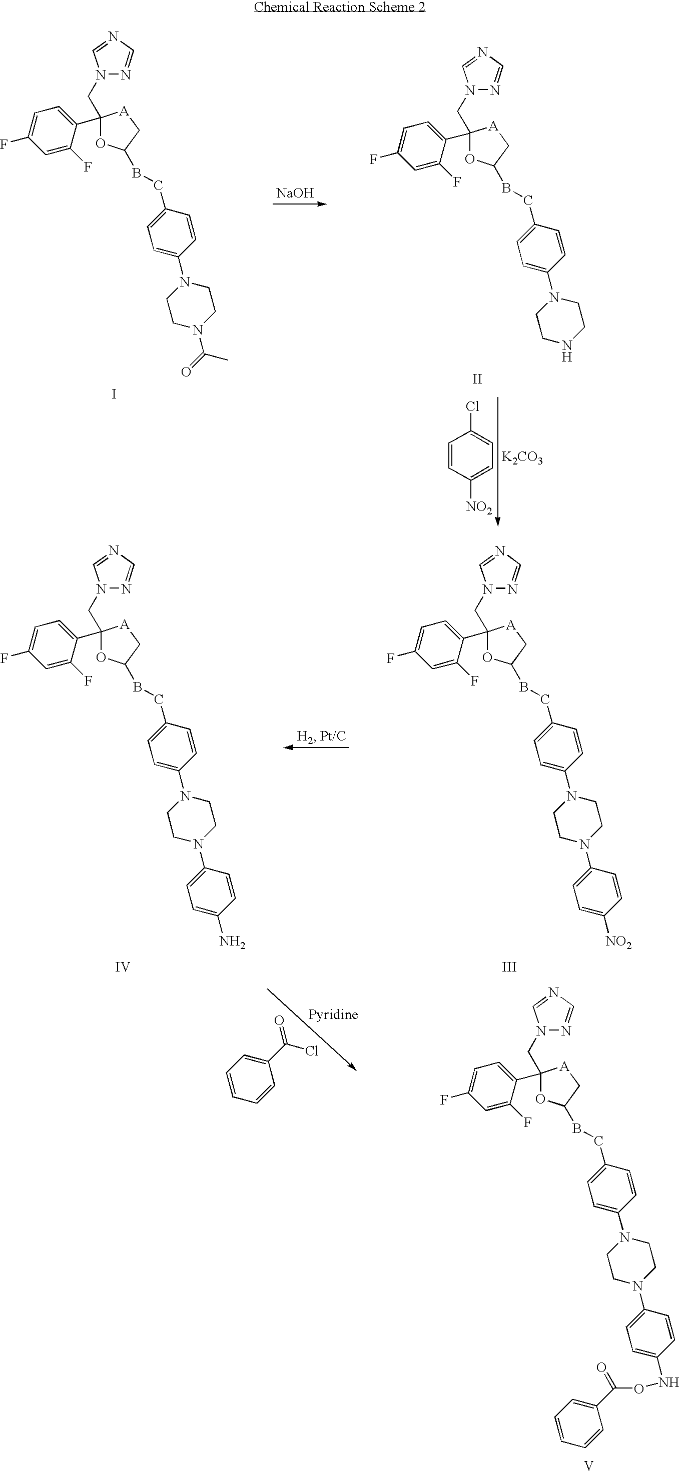

Reaction of (V) with hydrazine provided hydrazinecarboxamide (VI). The cyclization of (VI) in the presence of formamidine in hot DMF yielded the substituted triazolone (VII).

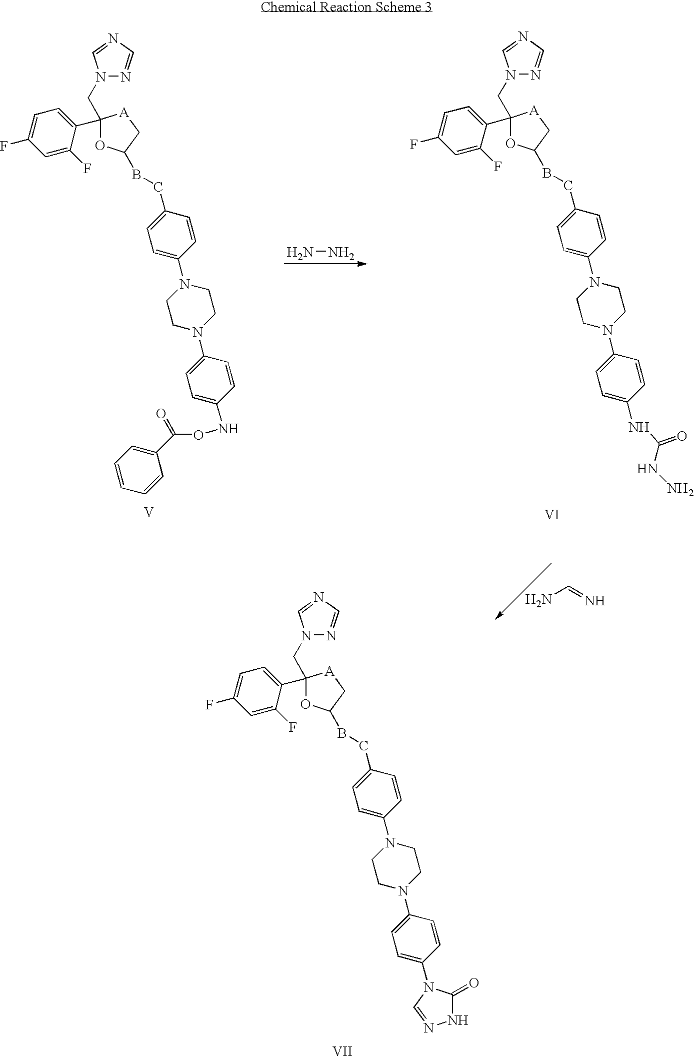

### Example 13

### Synthesis of 4-(4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-1-sec-butyl-1H-1,2,4-triazol-5(4H)-one (Formula I(D)-Rₐ)

2-brombutane (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (49%).

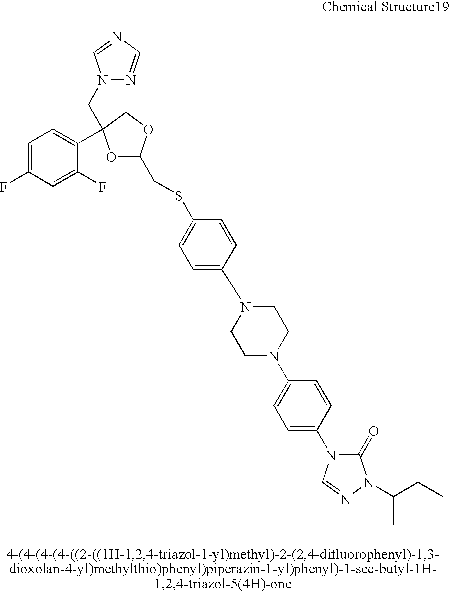

### Example 14

### Synthesis of 4-(4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-1-(pentan-3-yl)-1H-1,2,4-triazol-5(4H)-one (Formula I(D)-R_{b})

3-bromopentane (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-1H-1,2,4-triazol-5(4H)-one which was prepared from the Example 12 (1.0 mol) in 250 mL DMSO/MgSO4. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (54%).

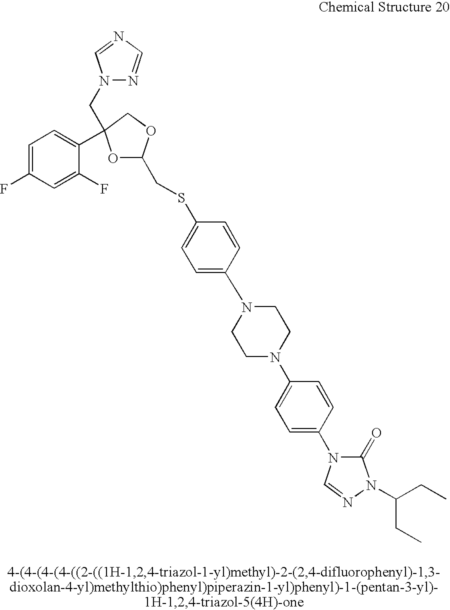

### Example 15

### Synthesis of 4-(4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-1-isopropyl-1H-1,2,4-triazol-5(4H)-one (Formula I(D)-R_{c})

2-bromopropane (1.2 mol) was added to a suspension of KOH powder (1.3 mol) and 4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (54%).

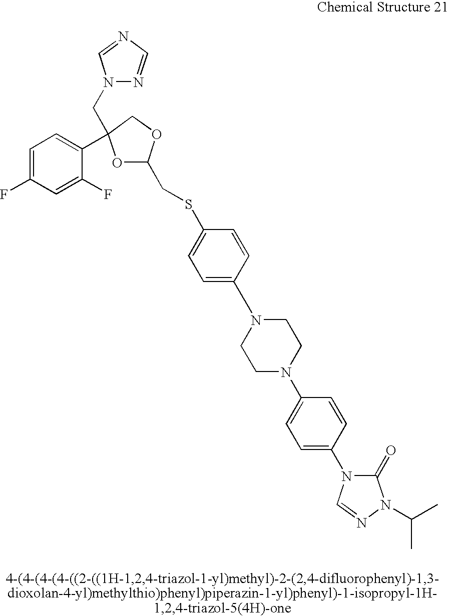

### Example 16

### Synthesis of 2-(4-(4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-5-oxo-4,5-dihydro-1H-1,2,4-triazol-1-yl)propanenitrile (Formula I(D)-R_{d})

2-bromopropanenitrile (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (47%).

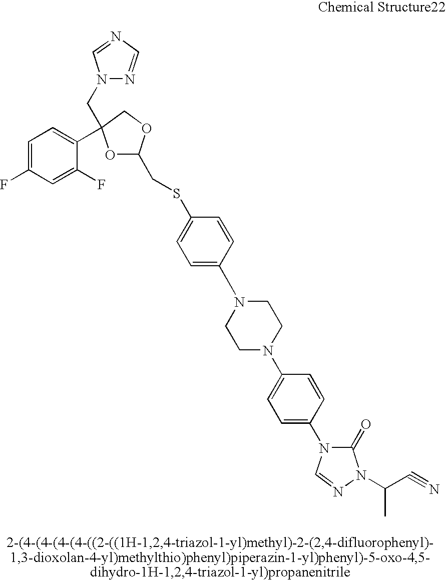

### Example 17

### Synthesis of 4-(4-(4-(4-((4-((1H-1,2,4-triazol-1-yl)methyl)-4-(2,4-difluorophenyl)-1,3-dioxolan-2-yl)methylthio)phenyl)piperazin-1-yl)phenyl)-1-(3-hydroxybutan-2-yl)-1H-1,2,4-triazol-5(4H)-one (Formula I(D)-Rₑ)

3-bromobutan-2-ol (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (47%).

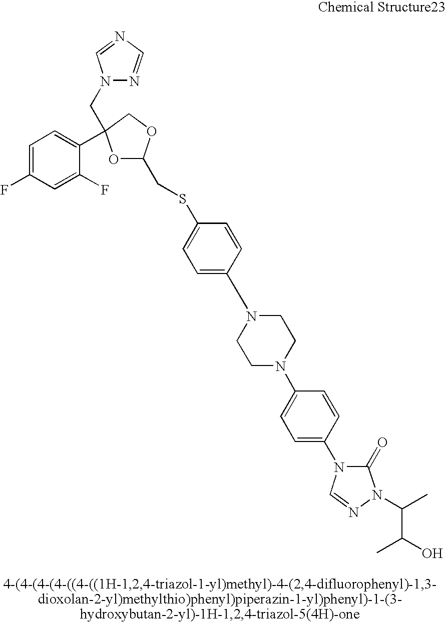

### Example 18

### Synthesis of 4-(4-(4-(4-((4-((1H-1,2,4-triazol-1-yl)methyl)-4-(2,4-difluorophenyl)-1,3-dioxolan-2-yl)methylthio)phenyl)piperazin-1-yl)phenyl)-1-(2-hydroxypentan-3-yl)-1H-1,2,4-triazol-5(4H)-one (Formula I(D)-R_{f})

3-bromopentan-2-ol (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (56%).

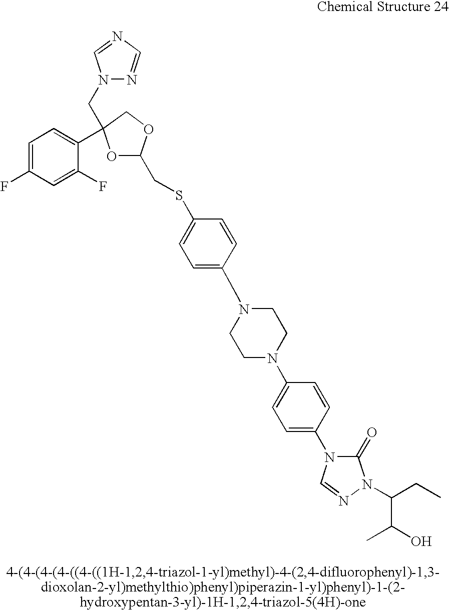

### Example 19

### Synthesis of 4-(4-(4-(4-((4-((1H-1,2,4-triazol-1-yl)methyl)-4-(2,4-difluorophenyl)-1,3-dioxolan-2-yl)methylthio)phenyl)piperazin-1-yl)phenyl)-1-(but-3-en-2-yl)-1H-1,2,4-triazol-5(4H)-one (Formula I(D)-R_{g})

3-bromobut-1-ene (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (56%).

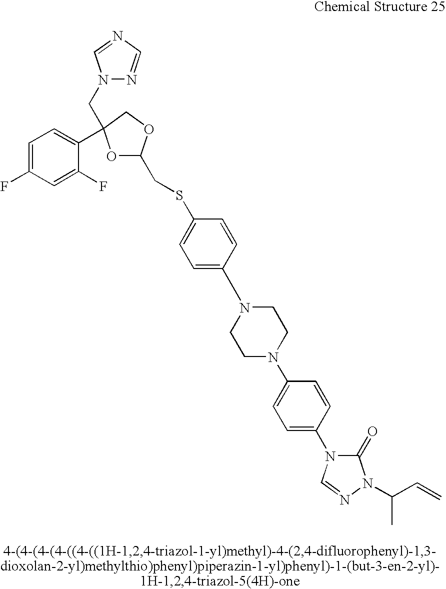

### Example 20

### Synthesis of 4-(4-(4-(4-((4-((1H-1,2,4-triazol-1-yl)methyl)-4-(2,4-difluorophenyl)-1,3-dioxolan-2-ylthio)methyl)phenyl)piperazin-1-yl)phenyl)-1-sec-butyl-1H-1,2,4-triazol-5(4H)-one (Formula II(D)-Rₐ)

2-brombutane (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. The product was further purified by recrystallization in toluene (49%).

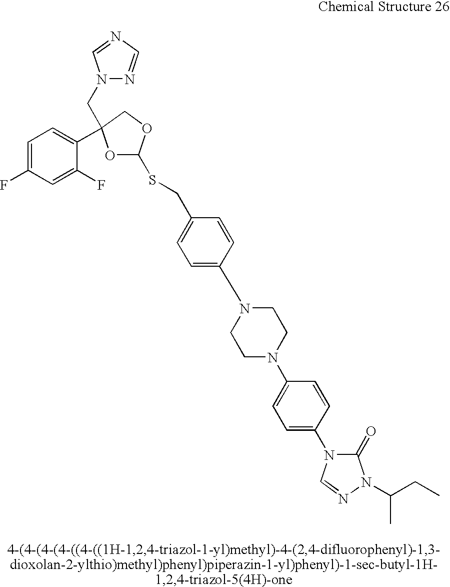

### Example 21

### Synthesis of 4-(4-(4-(4-((2-((1H-1,2,4-triazol-1-yl)methyl)-2-(2,4-difluorophenyl)-1,3-dioxolan-4-ylhio)mthyl)phenyl)piperazin-1-yl)phenyl)-1-(pentan-3-yl)-1H-1,2,4-triazol-5(4H)-one (Formula II(D)-R_{b})

3-bromopentane (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. And further purified by recrystallization in toluene (54%).

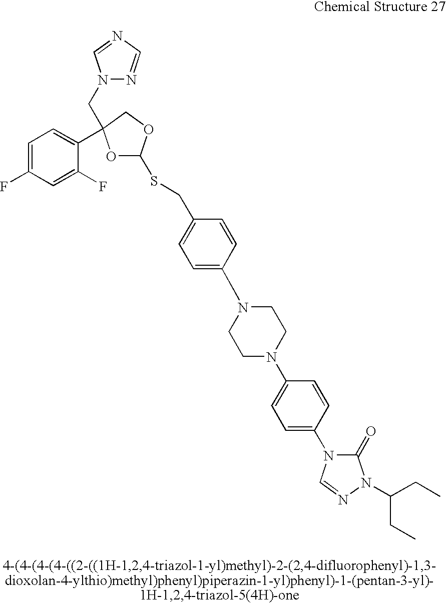

### Example 22

### Synthesis of 4-(4-(4-(4-(4((2-((1H-1,2,4-triazol-1-yl)methyl)-2-(2,4-difluorophenyl)-1,3-dioxolan-4-ylthio)methyl)phenyl)piperazin-1-yl)phenyl)-1-isopropyl-1H-1,2,4-triazol-5(4H)-one (Formula II(D)-R_{c})

2-bromopropane (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO4, solvent was evaporated under vacuo. The crude product was initially purified by flash chromatography with CHCl₃/CH₃OH (98/2) as the eluent and further purified by recrystallization in toluene (54%).

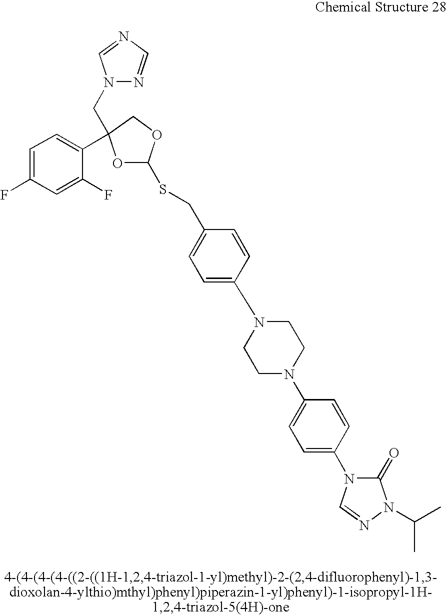

### Example 23

### Synthesis of 2-(4-(4-(4-(4-((2-((1H-1,2,4-triazol-1-yl)methyl)-2-(2,4-difluorophenyl)-1,3-dioxolan-4-ylthio)methyl)phenyl)piperazin-1-yl)phenyl)-5-oxo-4,5-dihydro-1H-1,2,4-triazol-1-yl)propanenitrile (Formula II(D)-R_{d})

2-bromopropanenitrile (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent, and further purified by recrystallization in toluene (47%).

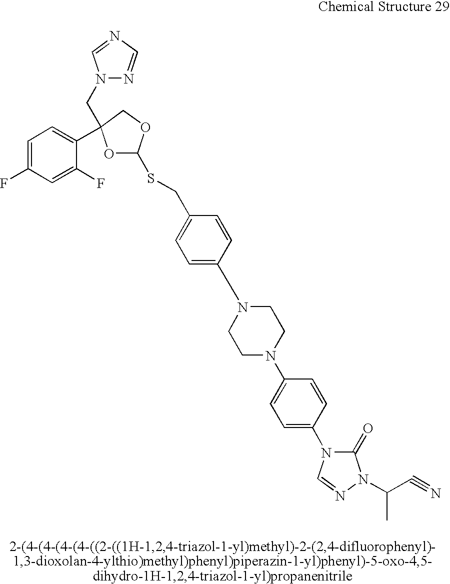

### Example 24

### Synthesis of 4-(4-(4-(4-((2-((1H-1,2,4-triazol-1-yl)methyl)-2-(2,4-difluorophenyl)-1,3-dioxolan-4-ylthio)methyl)phenyl)piperazin-1-yl)phenyl)-1-(3-hydroxybutan-2-yl)-1H-1,2,4-triazol-5(4H)-one (Formula II(D)-Rₑ)

3-bromobutan-2-ol (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (56%).

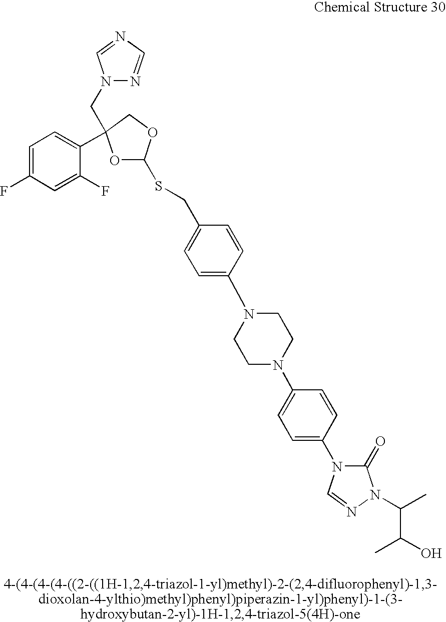

### Example 25

### Synthesis of 4-(4-(4-(4-((4-((1H-1,2,4-triazol-1-yl)methyl)-4-(2,4-difluorophenyl)-1,3-dioxolan-2-ylthio)methyl)phenyl)piperazin-1-yl)phenyl)-1-(2-hydroxypentan-3-yl)-1H-1,2,4-triazol-5(4H)-one (Formula II(D)-R_{f})

3-bromopentan-2-ol (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (56%).

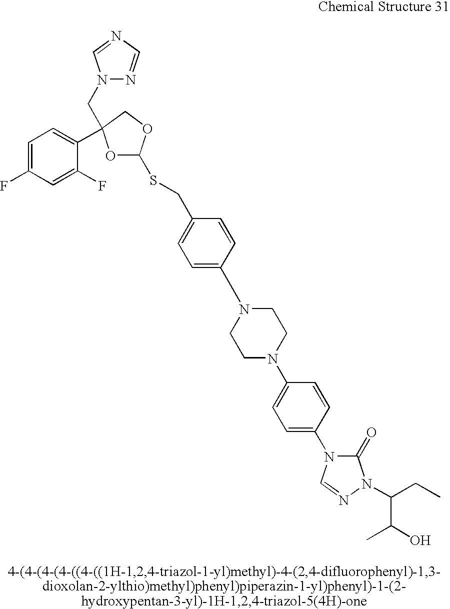

### Example 26

### Synthesis of 4-(4-(4-(4-((4-((1H-1,2,4-triazol-1-yl)methyl)-4-(2,4-difluorophenyl)-1,3-dioxolan-2-ylthio)methyl)phenyl)piperazin-1-yl)phenyl)-1-(but-3-en-2-yl)-1H-1,2,4-triazol-5(4H)-one (Formula II(D)-R_{g})

3-bromobut-1-ene (1.2 mol) was added to a suspension of KOH powder (1.3 mol) and 4-(4-(4-(4-((2-((1H-1,2,4-triazol-1-yl)methyl)-2-(2,4-difluorophenyl)-1,3-dioxolan-4-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (56%).

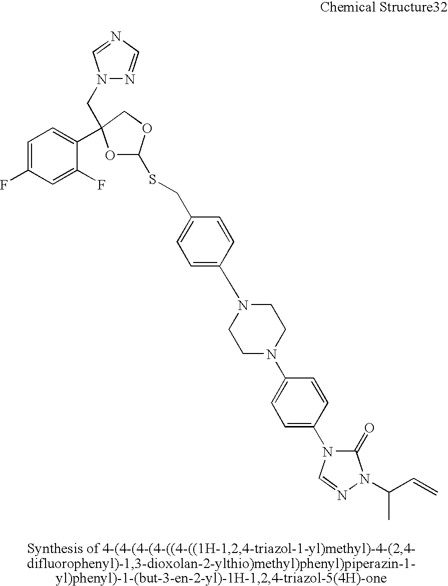

### Example 27

### Synthesis of 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1-sec-butyl-1H-1,2,4-triazol-5(4H)-one (Formula I(T)-Rₐ)

2-brombutane (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (49%).

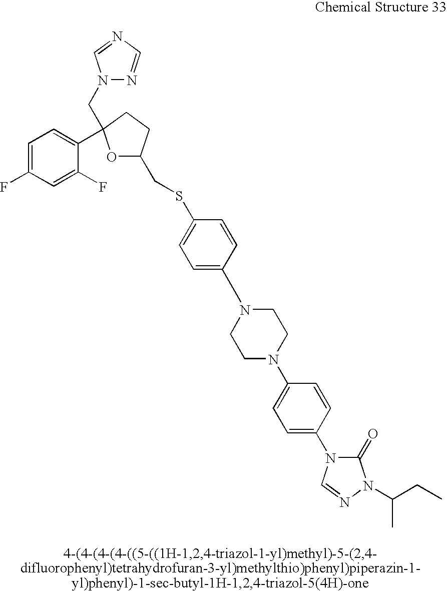

### Example 28

### Synthesis of 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1-(pentan-3-yl)-1H-1,2,4-triazol-5(4H)-one (Formula I(T)-R_{b})

3-bromopentane (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (54%).

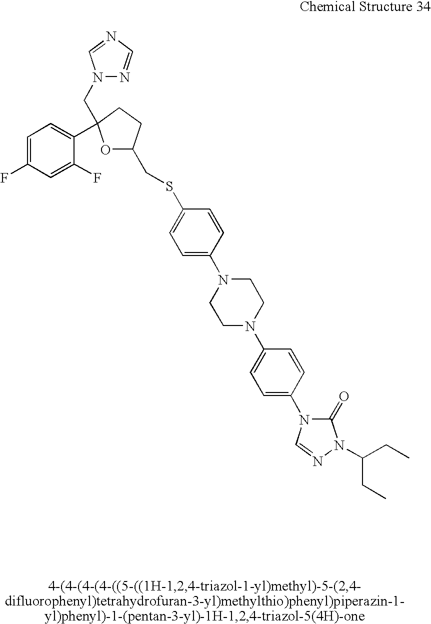

### Example 29

### Synthesis of 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1-isopropyl-1H-1,2,4-triazol-5(4H)-one (Formula I (T)-R_{c})

2-bromopropane (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by a flash chromatography with CHCl₃/CH₃OH (98/2) as the eluent. The product was further purified by recrystallization in toluene (54%).

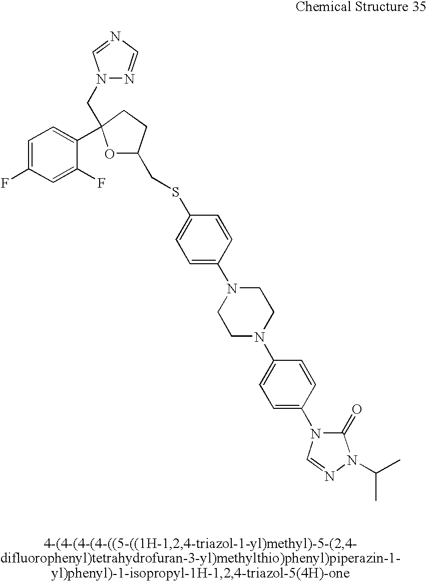

### Example 30

### Synthesis of 2-(4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-5-oxo-4,5-dihydro-1H-1,2,4-triazol-1-yl)propanenitrile (Formula I (T)-R_{d})

2-bromopropanenitrile (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. The product was further purified by recrystallization in toluene (47%).

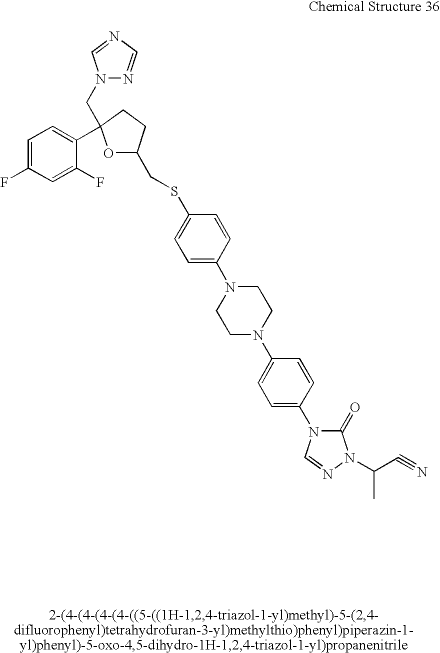

### Example 31

### Synthesis of 4-(4-(4-(4-((5-((1H-1,2,4-triazol-1-yl)methyl)-5-(2,4-difluorophenyl)tetrahydrofuran-2-yl)methylthio)phenyl)piperazin-1-yl)phenyl)-1-(3-hydroxybutan-2-yl)-1H-1,2,4-triazol-5(4H)-one (Formula I (T)-Rₑ)

3-bromobutan-2-ol (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. The product was further purified by recrystallization in toluene (56%).

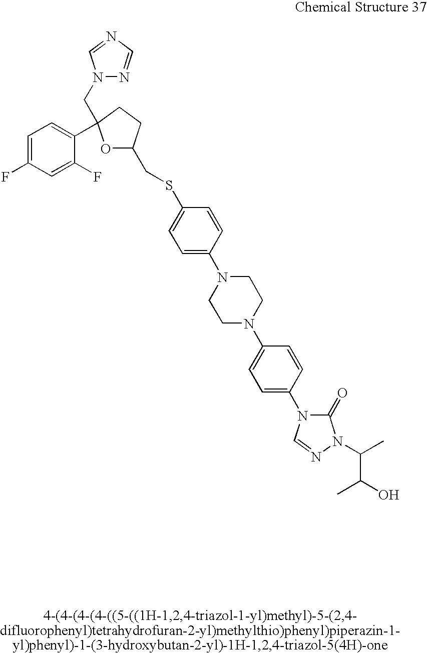

### Example 32

### Synthesis of 4-(4-(4-(4-((5-((1H-1,2,4-triazol-1-yl)methyl)-5-(2,4-difluorophenyl)tetrahydrofuran-2-yl)methylthio)phenyl)piperazin-1-yl)phenyl)-1-(2-hydroxypentan-3-yl)-1H-1,2,4-triazol-5(4H)-one (Formula II (T)-R_{f})

3-bromopentan-2-ol (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1H-1,2,4-triazol-5(4H)-one which was prepared from the Example 12 (1.0 mol) in 250 mL DMSO/MgSO4. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. The product was further purified by recrystallization in toluene (56%).

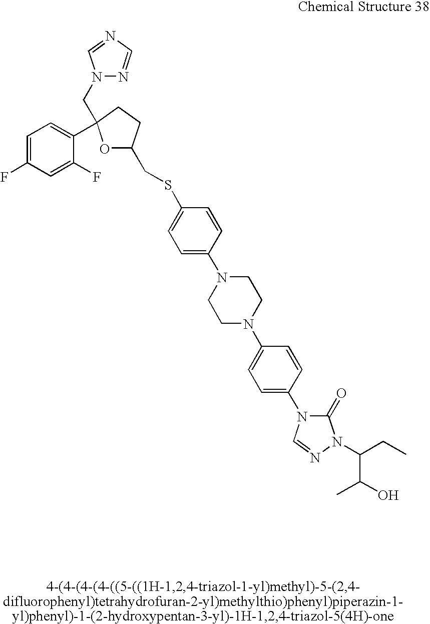

### Example 33

### Synthesis of 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1-(but-3-en-2-yl)-1H-1,2,4-triazol-5(4H)-one (Formula I (T)-R_{g})

3-bromobut-1-ene (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-yl)methylthio)phenyl)piperazin-1-yl)phenyl)-1H-1,2,4-triazol-5(4H)-one produced which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. The product was further purified by recrystallization in toluene (56%).

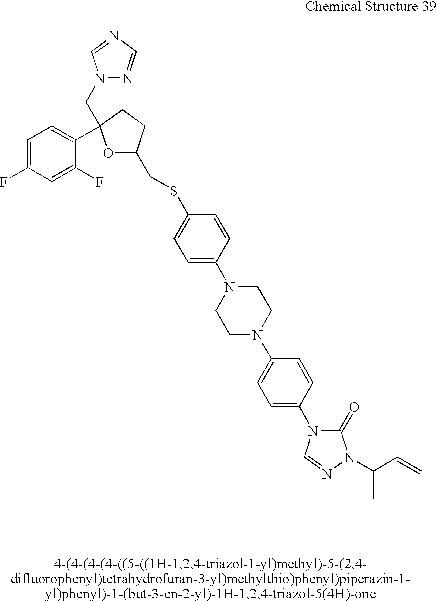

### Example 34

### Synthesis of 4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-1-sec-butyl-1H-1,2,4-triazol-5(4H)-one (Formula II (T)-Rₐ)

2-brombutane (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. The product was further purified by recrystallization in toluene (49%).

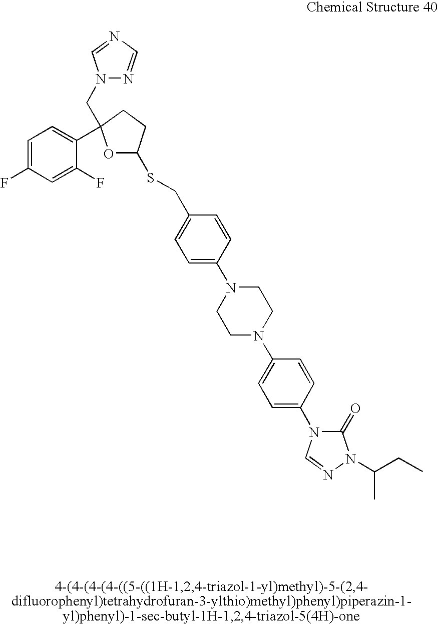

### Example 35

### Synthesis of 4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-1-(pentan-3-yl)-1H-1,2,4-triazol-5(4H)-one (Formula II-T R_{b})

3-bromopentane (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent and further purified by recrystallization in toluene (54%).

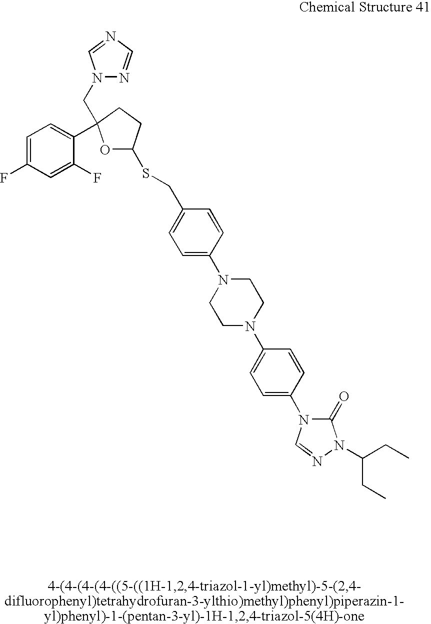

### Example 36

### Synthesis of 4-(4-(4-(4-((5-(1H-1,2,4-triazol-1-yl)methyl)-5-(2,4-difluorophenyl)tetrahydrofuran-3-ylthio)methyl)phenyl)piperazin-1-yl)phenyl)-1-isopropyl-1H-1,2,4-triazol-5(4H)-one (Formula II-T-R_{c})

2-bromopropane (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. The product was further purified by recrystallization in toluene (54%).

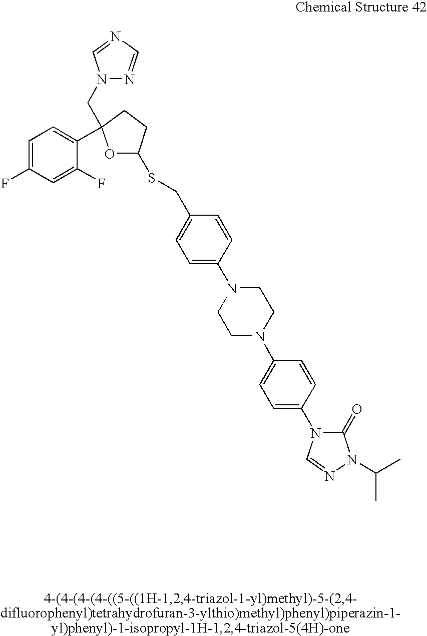

### Example 37

### Synthesis of 2-(4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-5-oxo-4,5-dihydro-1H-1,2,4-triazol-1-yl)propanenitrile (Formula II (T)-R_{d})

2-bromopropanenitrile (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. The product was further purified by recrystallization in toluene (47%).

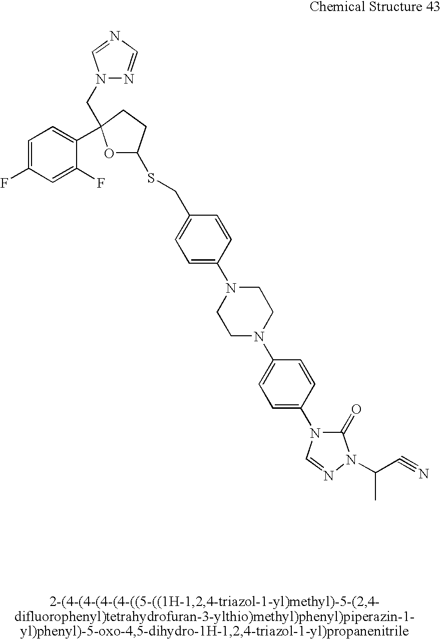

### Example 38

### Synthesis of 4-(4-(4-(4-((5-((1H-1,2,4-triazol-1-yl)methyl)-5-(2,4-difluorophenyl)tetrahydrofuran-2-ylthio)methyl)phenyl)piperazin-1-yl)phenyl)-1-(3-hydroxybutan-2-yl)-1H-1,2,4-triazol-5(4H)-one (Formula II-T-Rₑ)

3-bromobutan-2-ol (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. The product was further purified by recrystallization in toluene (56%).

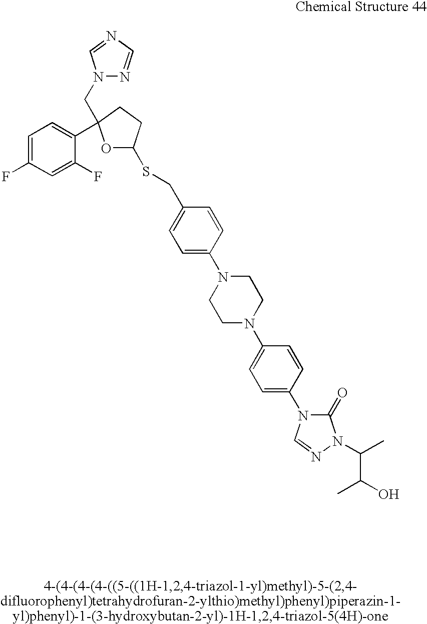

### Example 39

### Synthesis of 4-(4-(4-(4-((5-((1H-1,2,4-triazol-1-yl)methyl)-5-(2,4-difluorophenyl)tetrahydrofuran-2-ylhio)methyl)phenyl)piperazin-1-yl)phenyl)-1-(2-hydroxypentan-3-yl)-1H-1,2,4-triazol-5(4H)-one (Formula II-T-R_{f})

3-bromopentan-2-ol (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent. The product was further purified by recrystallization in toluene (56%).

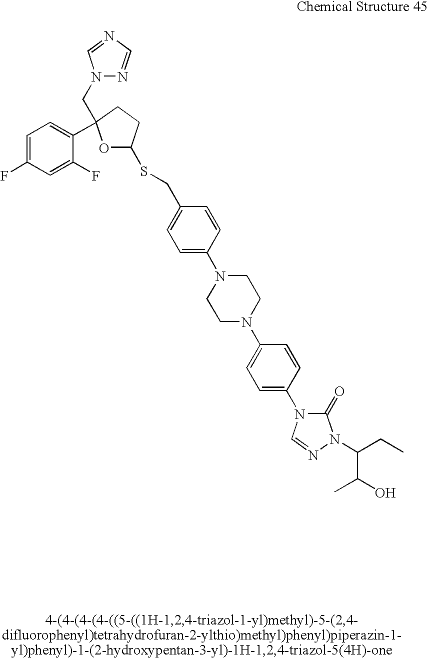

### Example 40

### Synthesis of 4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-1-(but-3-en-2-yl)-1H-1,2,4-triazol-5(4H)-one (Formula II-T-R_{g})

3-bromobut-1-ene (1.2 mol) was added to a suspension of KOH powder (1.3 mol) and 4-(4-(4-(4-((5-((1H-1,2,4-triazol-1-yl)methyl)-5-(2,4-difluorophenyl)tetrahydrofuran-3-ylthio)methyl)phenyl)piperazin-1-yl)phenyl)-1H-1,2,4-triazol-5(4H)-one which was prepared from the Example 12 (1.0 mol) in 250 mL DMSO/MgSO₄. The reaction mixture was stirred for 14 hrs, then was diluted with water. After extraction with CHCl₃ and dried over MgSO₄, solvent was evaporated under vacuo. The crude product was initially purified by flash chromatography with CHCl₃/CH₃OH (98/2) as the eluent, and further purified by recrystallization in toluene (56%).

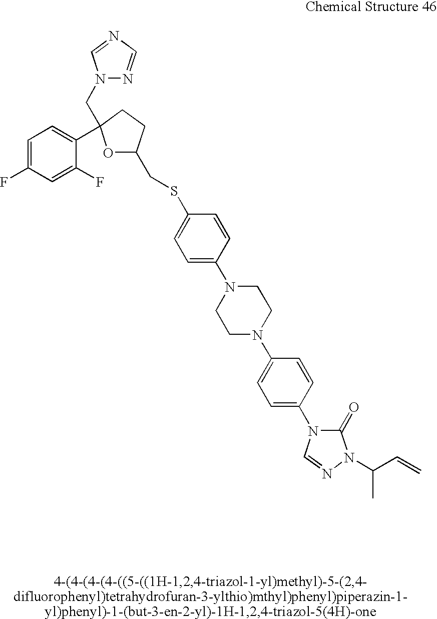

### Example 41

### In Vitro Activity Test

Organisms listed in Table 4 were tested according to an agar dilution method: Suspensions of each microorganism were prepared to contain 105 colony forming units (cfu)/mL. All drugs were dissolved in a few drops of DMSO then diluted with ethanol—water (1/1, v/v) to make a stock solution of 500 μg/mL. The agar dilution method was performed in a medium of Kimmig's agar (K. A., Merck) −0.5% glycerol [R. A. Fromtling, G. K. Abruzzo and A. Ruiz, Mycopathologia, 106 (1989) 163-166]. Plates of Kimmig's agar containing serial dilutions (25 to 0.01 μg/mL) of the drugs were inoculated with 10 μL of the fungal inocula and incubated at 25° C. during days for yeasts and up to 5 days for filamentous fungi. Following incubation, GMMICs (geometric mean minimum inhibitory concentration μg/mL) were determined. The results are shown in Table 4. In the table POCZ indicates posaconazole, ITRZ indicates itraconazole, and FLUZ indicates fluconazole. [Patterson, T. F., S. G. Revankar, W. R. Kirkpatrick, O. Dib, A. W. Fothergill, S. W. Redding, D. A. Sutton, and M. G. Rinaldi, J. Clin. Microbiol. 34 (1996) 1794-1797].

TABLE 4

| | | GMMICS (μg/mL) | | | |
|---|---|---|---|---|---|
| | No. | Compound I- | | | |
| Organism | Organism | D-Rₐ | POCZ | ITRZ | FLUZ |
| Aspergillus Flavus | 9 | 0.09 | 0.12 | 0.35 | >235 |
| Candida Krusei | 22 | 0.15 | 0.21 | 0.60 | 65 |
| Cryptococcus | 10 | 0.25 | 0.24 | 0.49 | 45 |
| neoformans | | | | | |
| Trichophyton | 17 | 0.10 | 1.2 | 3.1 | 105 |
| rubrum | | | | | |
| Microsporum canis | 6 | 0.35 | 0.50 | 1.2 | 151 |

Compounds of the invention were tested for their ability to inhibit ergosterol biosynthesis. Testing was performed in 96-well round-bottom microtitration plates. Cell suspensions were prepared in RPMI-1640 medium and were adjusted to give a final inoculum concentration of 0.5×10³ to 2.5×10³ cells/ml. The plates were incubated incubated at 30° C. for 48 h before growth was assessed. The MIC of each compounds was defined as the lowest concentration at which there was 80% inhibition of growth compared with that in a drug-free control [O. N. Breivik and J. L. Owades, Agric. Food Chem., 5 (1957) 360-363; T. F. Patterson, S. G. Revankar, W. R. Kirkpatrick, O. Dib, A. W. Fothergill, S. W. Redding, D. A. Sutton and M. G. Rinaldi, J. Clin. Microbiol., 34 (1996) 1794-1797]. Ergosterol content was calculated as a percentage of the wet weight of the cell [National Committee for Clinical Laboratory Standards. 1997. Reference method for broth dilution antifungal susceptibility testing of yeasts, Approved standard. Document M27-A, National Committee for Clinical Laboratory Standards, Wayne, Pa.].

TABLE 5
IC50 Values for Inhabitation of Ergosterol Biosynthesis

| | |
|---|---|
| Candida | Aspergillus |
| albicans_C286 | fumigatus_ND 158 |

| Agent | (nM) | Agent | (nM) |
|---|---|---|---|
| ITRZ | 39.1 | POCZ | 11.8 |
| I-Rₐ (T)* | 36.8 | I-Rₐ (T)* | 11.1 |
| I-R_{b} (T) | 39.8 | I-R_{b} (T) | 12.0 |
| I-Rₐ (D)** | 31.9 | I-Rₐ (D)** | 9.6 |
| I-R_{b} (D) | 32.7 | I-R_{b} (D) | 9.9 |
| I-R_{d} (D) | 33.1 | I-R_{d} (T) | 10.0 |
| I-Rₑ (D) | 31.9 | I-Rₑ (T) | 9.6 |
| I-R_{f} (T) | 32.1 | I-R_{f} (T) | 9.5 |
| I-R_{f} (D) | 33.4 | I-R_{f} (D) | 9.9 |
| I-R_{g} (D) | 33.8 | I-R_{g} (D) | 10.2 |
| II-Rₐ (T) | 34.3 | II-Rₐ(T) | 10.3 |
| II-Rₐ(D) | 34.3 | II-Rₐ (D) | 10.3 |
| II-R_{f} (T) | 32.9 | II-R_{f} (T) | 9.3 |
| II-R_{f} (D) | 31.1 | II-R_{f} (D | 9.8 |

These in vitro studies demonstrated favorable or comparable activity for Equaconazole when compared to posaconazole, itraconazole, and fluconazole against a variety of fungal pathogens. Furthermore, equaconazole was shown to demonstrate favorable antifungal activity against Ergosterol Biosynthesis. Based on the in intro antifungal activity, preferred compounds within the present invention are I-Rₐ (T) and (D), I-R_{b} (D), I-R_{d} (T) and (D), I-Rₑ (T) and (D), I-R_{f} (T) and (D), I-R_{g} (T) and (D), II-Rₐ (T) and (D), II-R_{f} (T) and (D). More preferred compounds are I-Rₐ (T) and (D), I-R_{d} (T) and (D), I-Rₑ (T) and (D), I-R_{f} (T) and (D), II-Rₐ (T) and (D), II-R_{f} (T) and (D). Particularly preferred compounds are I-Rₐ (T) and (D), I-R_{f} (T) and (D), II-Rₐ (T) and (D), II-R_{f} (T) and (D).

### Example 42

### Antifungal Oral Solution

A suspension suitable for oral delivery of equaconazoles is prepared. DAG-PEG was added to a vessel equipped with a mixer propeller. The drug substance was added with constant mixing. Mixing continued until the drug was visually dispersed in the lipids. Pre-dissolved excipients in water were slowly added to the vessel with adequate mixing. Mixing continued until fully a homogenous solution was achieved. A sample formulation is described in Table 6.

TABLE 6

| Ingredient | mg/mL |
|---|---|
| Equaconazole | 20.0 |
| PEG Lipid | 60 |
| Organic Acid | 25 |
| Sodium Hydroxide | See below |
| Hydrochloric Acid | See below |
| Sodium Benzoate | 2.0 |
| Artificial Flavor | 5.0 |
| Purified Water | qs 1 mL |

The lipid may be PEG-12-N₃-GDO, PEG-12-N₃-GDM, PEG-12-N₃-GDLO, PEG-12-N₃-GDP, PEG-12-Ac₂-GDO, PEG-12-Ac₂-GDM or any combination thereof. Sodium hydroxide is used to prepare a 10% w/w solution in purified water. The targeted pH is in a range of 4.0 to 7.0. The NaOH solution is used to adjust pH if necessary. The drug to lipid ratio is preferably greater than about 1 to 20, and more preferably greater than about 1 to 5. The organic acid may be lactic acid or pyruvic acid or glycolic acid, though lactic acid is most preferable. The concentration of organic acid is preferably in the range 1 and 10%, and more preferably about 2 to 5%.

### Example 43

### Antifungal IV Injectable Solution

The IV solution was prepared as in Example 20, except that the targeted pH range was between 6.5 and 7.5. A sample formulation is described in Table 7.

TABLE 7

| Ingredient | mg/mL |
|---|---|
| Equaconazole | 10.0 |
| DAG-PEG Lipid | 50 |
| Sodium Hydroxide | See Below |
| Lactic Acid | 25 |
| Purified Water | qs 1 mL |

The lipid may be PEG-12-N₃-GDO, PEG-12-N₃-GDM, PEG-12-N₃-GDLO, PEG-12-N₃-GDP, PEG-12-Ac₂-GDO, PEG-12-Ac₂-GDM or any combination thereof. Sodium hydroxide is used to prepare a 10% w/w solution in purified water. The targeted pH is in a range of 6.5 to 7.0. The NaOH solution is used to adjust pH if necessary.

### Example 44

### Pharmacokinetic Profile and Bioavailability of Antifungal Formulations

Experiments were performed to determine blood levels of equaconazole formulations after both intravenous and oral dosing. For comparison, posaconazole formulations were also tested. Groups of three male mice (B6D2F1) were used for the studies. HPLC-MS analyses were performed on heparinized mouse plasma samples obtained typically at 0 hr, 0.08 hr, 0.25 hr, 0.5 hr, 1 hr, 2 hr, 4 hr, 8 hr, 16 hr and 24 hr after bolus IV injection. After oral feeding, the anises were performed at 0 hr, 0.08 hr, 0.25 hr, 0.5 hr, 1 hr, 2 hr, 4 hr, 8 hr, 16 hr and 24 hr. To determine the level of each drug, the drug was first isolated from plasma with a sample pre-treatment. Acetonitrile were used to remove proteins in samples. An isocratic HPLC-MS method was then used to separate the drugs from any potential interference. Drug levels were measured by MS detection with a multiple reaction monitoring (MRM) mode. PK data was analyzed using the WinNonlin program (ver. 5.2, Pharsight) compartmental models of analysis. The results demonstrated that formulations of compounds in the present invention have a superior PK profile than posaconazole.

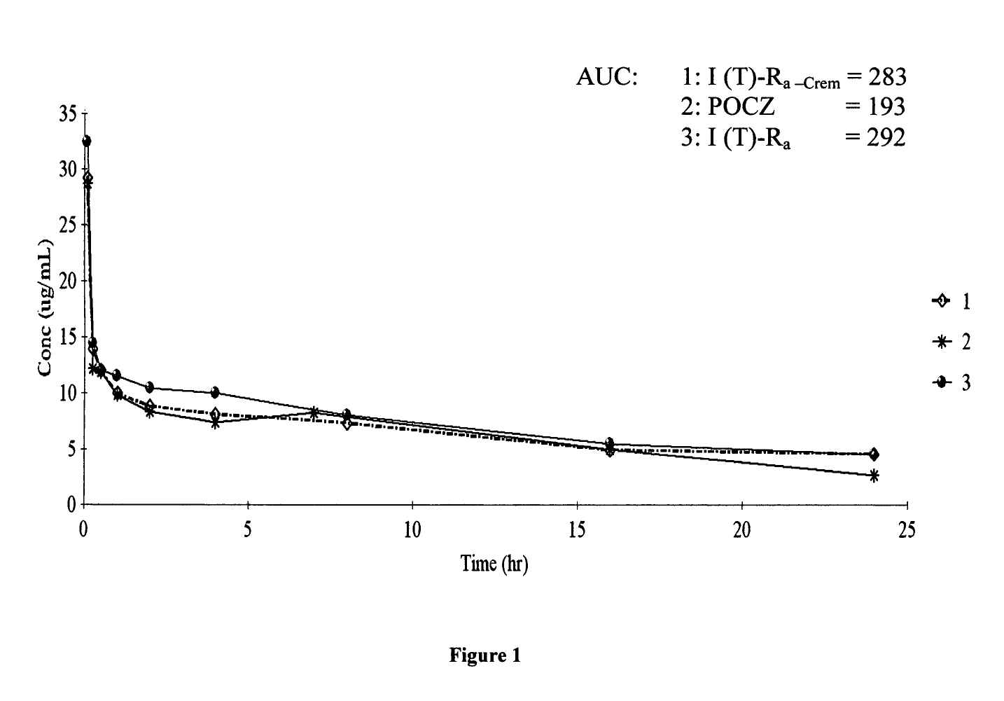

FIG. 1 shows mouse PK profiles of posaconazole formulations with (1) Compound I(T)-Rₐ solution containing 5% dimethyl sulfoxide and 10%_Cremophor (I(T)-R_{a-Crem}) (2) posaconazole solution containing 5% dimethyl sulfoxide and 10% Cremophor (POCZ) and (3) Formula I (T)-Rₐ in 5% DAG-PEG (GDO-12, 1,2-dioleoyl-rac-glycerol-3-dodecaethylene glycol) liposomal formulation (I(D)-Rₐ). The drug was administered intravenously and the dosing strength was 10 mg/kg. The AUC were 283 μg·hr/mL and 193 μg hr/mL and 292 μg·hr/mL for I(T)-R_{a-Crem}, POCZ and I(T)-Rₐ, respectively.

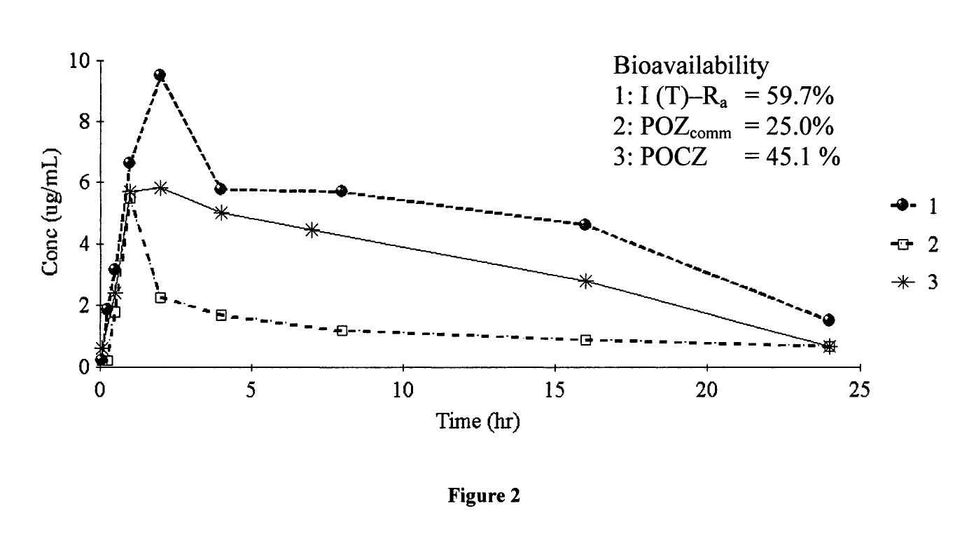

FIG. 2 shows mouse PK profiles of (1) Compound I(T)-Rₐ in 5% DAG-PEG (GDO-12, 1,2-dioleoyl-rac-glycerol-3-dodecaethylene glycol), (2) commercial product (POZ_{comm}) of Posaconazole suspension (Noxafil®, Schering-Plough) and (3) posaconazole in 5% DAG-PEG (GDO-12, 1,2-dioleoyl-rac-glycerol-3-dodecaethylene glycol) liposomal formulation (POCZ). The drug was administered orally and the dosing strength was 10 mg/kg. The relative bioavailabilities were 59.7%, 25.0% and 45.1% for I(T)-Rₐ, POZ_{comm} and POCZ, respectively.

### Example 45

### Antifungal Topical Cream

DAG-PEG lipid was added to a stainless steel vessel equipped with propeller type mixing blades. The drug substance was added with constant mixing. Mixing continued until the drug was visually dispersed in the lipids at a temperature to 60°-65° C. Organic acid, Cholesterol and glycerin were added with mixing. Ethanol and ethyoxydiglycol were added with mixing. Finally Carbopol ETD 2020, purified water and triethylamine were added with mixing. Mixing continued until fully a homogenous cream was achieved. The formulation is described in Table 8.

TABLE 8

| Ingredient | % |
|---|---|
| Equaconazole | 1.0 |
| PEG Lipid | 5.0 |
| Carbopol ETD 2020 | 0.5 |
| Ethyoxydiglycol | 1.0 |
| Ethanol | 5.0 |
| Glycerin | 1.0 |
| Cholesterol | 0.4 |
| Triethylamine | 0.20 |
| Organic acid | 5 |
| Sodium hydroxide | See below |
| Purified water | qs 100 |

The lipid may be PEG-12-N₃-GDO, PEG-12-N₃-GDM, PEG-12-N₃-GDLO, PEG-12-N₃-GDP, PEG-12-Ac₂-GDO, PEG-12-Ac₂-GDM or any combination thereof (see Example 21). Organic acid may be lactic acid or pyruvic acid or glycolic acid. Sodium hydroxide is used to adjust pH if necessary. The targeted pH range was between 3.5 and 7.0.

### Example 46

### Antifungal Topical Solution

The topical solution was prepared as in Example 23, except that active was first dissolved in organic acid and ethanol. A sample formulation is described in Table 9.

TABLE 9

| Ingredient | % |
|---|---|
| Equaconazole | 1.0 |
| PEG Lipid | 5.0 |
| α-Tocopherol | 0.5 |
| Organic acid | 2.5 |
| Ethanol | 5.0 |
| Sodium Benzoate | 0.2 |
| Sodium Hydroxide | See Below |
| Purified Water | qs 100 |

The lipid may be PEG-12-N₃-GDO, PEG-12-N₃-GDM, PEG-12-N₃-GDLO, PEG-12-N₃-GDP, PEG-12-Ac₂-GDO, PEG-12-Ac₂-GDM or any combination thereof. Organic acid may be lactic acid or pyruvic acid or glycolic acid (also see Example 21). Sodium hydroxide is used to adjust pH if necessary. The targeted pH range was between 3.5 and 7.0.

### Example 47

### Antifungal Capsules

A sample formulation is described in Table 10. Equaconazole was charged to a suitable vessel equipped with a mixer propeller. Lactic acid was added with gentle mixing to levigate the drug powder. 100% of the final batch volume of PEG-lipid was added with constant mixing. Mixing was continued until the suspension was fully dispersed. Vitamin E TPGS (D-alpha-tocopheryl polyethylene glycol 1000 succinate) was slowly added to the vessel with constant mixing. Mixing was continued with slow agitation (50 to 55° C.) until the Vitamin E TPGS was visually dispersed in the solution. The mixture was kept warm and transferred to the filling steps.

The appropriate filling equipment (e.g. Bosch's GKF 1400L) was set up with the required fill volume. The batch was filled into the capsules. The batch was continually agitated. No. 0 blue opaque hard gelatin capsule shells at a target fill weight of 550.0 mg were used, employing a suitable capsule machine (e.g., Bosch GKF 2000S Capsule filler or Capsugel CFS 1200 or Planeta Capsule Filler). The capsules were transferred into a suitable closed cool chamber container (0 to −20° C.) over night to let the capsule content be solidified. The solidified capsules were polished using a suitable polisher (e.g., Key Turbo Kleen CP-300 Capsule Polisher). The finished capsules were transferred into a suitable closed container.

TABLE 10

| Ingredient | mg/cap |
|---|---|
| Equaconazole Micronized | 100.0 |
| Lactic acid | 50 |
| PEG Lipid | 200.0 |
| Vitamin E TPGS | 200.0 |

The lipid may be PEG-12-N₃-GDO, PEG-12-N₃-GDM, PEG-12-N₃-GDLO, PEG-12-N₃-GDP, PEG-12-Ac₂-GDO, PEG-12-Ac₂-GDM or any combination thereof. Organic acid may be lactic acid or pyruvic acid or glycolic acid (also see Example 21).

We claim:

1. A compound represented by the formula 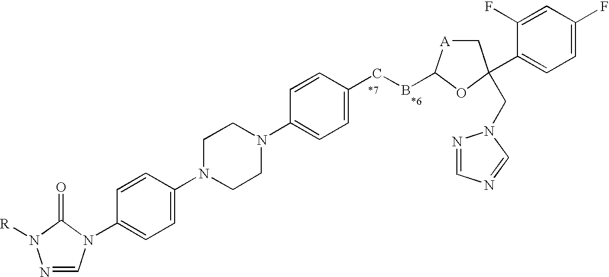
where A is CH₂ or oxyl, one of B and C is thiol and the other is CH₂, and R is selected from the group consisting of sec-butyl, isopentanyl, isopropyl, 2-isopropriono-1-nitrile, 3-isobutan-2-ol, 3-isopentan-2-ol, and 2-isobut-1-ene.

2. An ester of the compound of claim 1, where R is selected from the group consisting of 3-isobutan-2-ol, 3-isopentan-2-ol, such ester convertible in vivo into OH.

3. A pharmaceutically acceptable salt of the compound of claim 1, where R is selected from the group consisting of 3-isobutan-2-ol, 3-isopentan-2-ol, such salt convertible in vivo into OH.

4. A pharmaceutical composition for treating or preventing fungal infection comprising an antifungally effective amount of a compound of claim 1 together with a pharmaceutically acceptable carrier therefore.

5. The pharmaceutical composition of claim 4, where the pharmaceutically acceptable carrier is a DAG-PEG.

6. The pharmaceutical composition of claim 5, where the DAG-PEG is selected from the group consisting of PEG-12 GDO, PEG-12 GDM, PEG-23 GDP, PEG-12 GDS and PEG-23 GDS.

7. A method of treating and/or preventing a fungal infection in a mammal comprising administering an antifungally effective amount of a compound of claim 1 sufficient for such treating or preventing.

8. A method of treating and/or preventing a fungal infection in a mammal comprising administering an antifungally effective amount of a compound of claim 5 sufficient for such treating or preventing.

9. The method of claim 7, where such administering employs a means selected from the group consisting of oral capsule, oral solution, topical solution, and intravenous suspension.

10. A method of making a pharmaceutical composition for treating or preventing a fungal infective comprising combining a compound of claim 1 with a DAG-PEG and an aqueous solution to form liposomes.

11. The method of claim 8, where the DAG-PEG is selected from the group consisting of PEG-12 GDO, PEG-12 GDM, PEG-23 GDP, PEG-12 GDS and PEG-23 GDS.

12. A compound represented by the formula 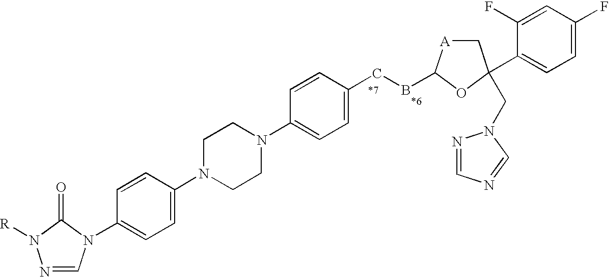
where A is CH₂ or oxyl, one of B and C is thiol and the other is CH₂, and R has a molecular weight below about 200.

13. An ester of the compound of claim 12, where R comprises and OH group.

14. A pharmaceutically acceptable salt of the compound of claim 12, where R comprises an OH group.

15. A pharmaceutical composition for treating or preventing fungal infection comprising an antifungally effective amount of a compound of claim 12 together with a pharmaceutically acceptable carrier therefore.

16. The pharmaceutical composition of claim 15, where the pharmaceutically acceptable carrier is a DAG-PEG.

17. The pharmaceutical composition of claim 16, where the DAG-PEG is selected from the group consisting of PEG-12 GDO, PEG-12 GDM, PEG-23 GDP, PEG-12 GDS, and PEG-23 GDS.
